SEQUENCE LISTING (69 sequences)

SEQ ID NO: 1 - protein, 7 aa, Canis familiaris
PSEKNSL

SEQ ID NO: 2 - protein, 20 aa, Canis familiaris
IHNCDPANPSEKNSLSIQYC

SEQ ID NO: 3 - protein, 20 aa, Homo sapiens
IYNCEPANPSEKNSPSTQYC

SEQ ID NO: 4 - protein, 21 aa, Felis catus
IHTCQPESKPSEKNSLSIKYC

SEQ ID NO: 5 - protein, 20 aa, Equus caballus
IYNCESANPAERNTLSIKYC

SEQ ID NO: 6 - protein, 20 aa, Mus musculus
IYDCEPSNSSEKNSPSTQYC

SEQ ID NO: 7 - protein, 23 aa, Artificial Sequence
  Caninised GA101 VK Kappa Light Chain variable domain FR1 framework sequence
DIVMTQTPLSLSVTPEEPASISC

SEQ ID NO: 8 - protein, 15 aa, Artificial Sequence
  Caninised GA101 VK Kappa Light Chain variable domain FR2 framework sequence
WYLQKPGQSPQLLIY

SEQ ID NO: 9 - protein, 32 aa, Artificial Sequence
  Caninised GA101 VK Kappa Light Chain variable domain FR3 framework sequence
GVPDRFSGSGSGTDFTLKISRVEAEDVGVYYC

SEQ ID NO: 10 - protein, 10 aa, Artificial Sequence
  Caninised GA101 VK Kappa Light Chain variable domain FR4 framework sequence
FGAGTKVEIK

SEQ ID NO: 11 - protein, 27 aa, Artificial Sequence
  Caninised GA101 VH Heavy Chain variable domain FR1 framework sequence
EVQLVQSGGEVVKPGGSLKVSCVASGF

SEQ ID NO: 12 - protein, 14 aa, Artificial Sequence
  Caninised GA101 VH Heavy Chain variable domain FR2 framework sequence
WVRQAPGQGMEWVG

SEQ ID NO: 13 - protein, 32 aa, Artificial Sequence
  Caninised GA101 VH Heavy Chain variable domain FR3 framework sequence
RVTITRDNSKSTAYLELSSLRSEDTAVYYCAR

SEQ ID NO: 14 - protein, 11 aa, Artificial Sequence
  Caninised GA101 VH Heavy Chain variable domain FR4 framework sequence
WGQGTLVTVSS

SEQ ID NO: 15 - protein, 112 aa, Artificial Sequence
  Complete caninised GA101 VK kappa variable light chain sequence
DIVMTQTPLSLSVTPEEPASISCRSSKSLLHSNGITYLYWYLQKPGQSPQLLIYQMSNLVSGVPDRFSGSGSGTDFTLKISRVEAEDVGVYYCAQNLELPYTFGAGTKVEIK

SEQ ID NO: 16 - protein, 119 aa, Artificial Sequence
  Complete caninised GA101 VH variable heavy chain sequence
EVQLVQSGGEVVKPGGSLKVSCVASGFAFSYSWINWVRQAPGQGMEWVGRIFPGDGDTDYNGKFKGRVTITRDNSKSTAYLELSSLRSEDTAVYYCARNVFDGYWLVYWGQGTLVTVSS

SEQ ID NO: 17 - protein, 454 aa, Artificial Sequence
  Complete caninised GA101 heavy chain (HCB isotype)
EVQLVQSGGEVVKPGGSLKVSCVASGYAFSYSWINWVRQAPGQGMEWVGRIFPGDGDTDYNGKFKGRVTITRDNSKSTAYLELSSLRSEDTAVYYCARNVFDGYWLVYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 18 - protein, 222 aa, Artificial Sequence
  Complete caninised GA-101 light chain
DIVMTQTPLSLSVTPEEPASISCRSSKSLLHSNGITYLYWYLQKPGQSPQLLIYQMSNLVSGVPDRFSGSGSGTDFTLKISRVEAEDVGVYYCAQNLELPYTFGAGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 19 - protein, 120 aa, Mus musculus
QVQLKQSGPGLVPPSQSLSITCTVSGFSLTNYGVHWVRQSPGKGLEWLGVIWSGGTTDYNAAFISRLSISKDNSKSQVFFKMNSLQADDTAIYYCARGPRKFYYYGMDYWGQGTSVTVSS

SEQ ID NO: 20 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAGDLGVYYCFQGLHVPLTFGAGTRLELK

SEQ ID NO: 21 - protein, 120 aa, Mus musculus
QVQLKQSGPGLVPPSQSLSITCTISGFSLTSYGVHWVRQSPGWGLEWLGVIWSGGSIDYNAAFISRLSISKDNSKSQVFIKMNSLQADDTAIYYCARGPRKFYYFGMDYWGQGTSVTVSS

SEQ ID NO: 22 - protein, 112 aa, Mus musculus
DVVMTQTPLSLPVSLGDQASISCRSSQNIVHSNGNTYVEWYLQKPGQSPKLLIYKVSNRFSGVPDRFSGSGSGTDFTLKISRVEAEDLGVYYCFQGSHVPLTFGAGTKLELK

SEQ ID NO: 23 - protein, 469 aa, Mus musculus
MAVLGLLFCLVTFPSCVLSQVQLKQSGPGLVPPSQSLSITCTVSGFSLTNYGVHWVRQSPGKGLEWLGVIWSGGTTDYNAAFISRLSISKDNSKSQVFFKMNSLQADDTAIYYCARGPRKFYYYGMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 24 - protein, 238 aa, Mus musculus
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAGDLGVYYCFQGLHVPLTFGAGTRLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - protein, 469 aa, Mus musculus
MAVLGLLFCLVTFPSCVLSQVQLKQSGPGLVPPSQSLSITCTISGFSLTSYGVHWVRQSPGWGLEWLGVIWSGGSIDYNAAFISRLSISKDNSKSQVFIKMNSLQADDTAIYYCARGPRKFYYFGMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 26 - protein, 238 aa, Mus musculus
MKLPVRLLVLMFWIPASNSDVVMTQTPLSLPVSLGDQASISCRSSQNIVHSNGNTYVEWYLQKPGQSPKLLIYKVSNRFSGVPDRFSGSGSGTDFTLKISRVEAEDLGVYYCFQGSHVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 27 - protein, 470 aa, Artificial Sequence
  Chimeric RA2 VH and canine HCA constant domain
MEWSWVFLFFLSVTTGVHSQVQLKQSGPGLVPPSQSLSITCTVSGFSLTNYGVHWVRQSPGKGLEWLGVIWSGGTTDYNAAFISRLSISKDNSKSQVFFKMNSLQADDTAIYYCARGPRKFYYYGMDYWGQGTSVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 28 - protein, 474 aa, Artificial Sequence
  Chimeric RA2 VH and canine HCB constant domain
MEWSWVFLFFLSVTTGVHSQVQLKQSGPGLVPPSQSLSITCTVSGFSLTNYGVHWVRQSPGKGLEWLGVIWSGGTTDYNAAFISRLSISKDNSKSQVFFKMNSLQADDTAIYYCARGPRKFYYYGMDYWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 29 - protein, 472 aa, Artificial Sequence
  Chimeric RA2 VH and canine HCC constant domain
MEWSWVFLFFLSVTTGVHSQVQLKQSGPGLVPPSQSLSITCTVSGFSLTNYGVHWVRQSPGKGLEWLGVIWSGGTTDYNAAFISRLSISKDNSKSQVFFKMNSLQADDTAIYYCARGPRKFYYYGMDYWGQGTSVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 30 - protein, 470 aa, Artificial Sequence
  Chimeric RA2 VH and canine HCD constant domain
MEWSWVFLFFLSVTTGVHSQVQLKQSGPGLVPPSQSLSITCTVSGFSLTNYGVHWVRQSPGKGLEWLGVIWSGGTTDYNAAFISRLSISKDNSKSQVFFKMNSLQADDTAIYYCARGPRKFYYYGMDYWGQGTSVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFYPPEIDVEWQSNGQPEPESKYHTTAPQLDEDGSYFLYSKLSVDKSRWQQGDTFTCAVMHEALQNHYTDLSLSHSPGK

SEQ ID NO: 31 - protein, 242 aa, Artificial Sequence
  Chimeric RA2 VL and canine kLC constant domain
MSVPTQVLGLLLLWLTDARCDVLMTQTPLSLPVSLGDQASISCRSSQSIVHSNGNTYLEWYLQKPGQSPKLLIYKVSNRFSGVPDRFSGSGSGTDFTLKISRVEAGDLGVYYCFQGLHVPLTFGAGTRLELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 32 - protein, 470 aa, Artificial Sequence
  Chimeric RF2 VH and canine HCA constant domain
MEWSWVFLFFLSVTTGVHSQVQLKQSGPGLVPPSQSLSITCTISGFSLTSYGVHWVRQSPGWGLEWLGVIWSGGSIDYNAAFISRLSISKDNSKSQVFIKMNSLQADDTAIYYCARGPRKFYYFGMDYWGQGTSVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 33 - protein, 474 aa, Artificial Sequence
  Chimeric RF2 VH and canine HCB constant domain
MEWSWVFLFFLSVTTGVHSQVQLKQSGPGLVPPSQSLSITCTISGFSLTSYGVHWVRQSPGWGLEWLGVIWSGGSIDYNAAFISRLSISKDNSKSQVFIKMNSLQADDTAIYYCARGPRKFYYFGMDYWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 34 - protein, 472 aa, Artificial Sequence
  Chimeric RF2 VH and canine HCC constant domain
MEWSWVFLFFLSVTTGVHSQVQLKQSGPGLVPPSQSLSITCTISGFSLTSYGVHWVRQSPGWGLEWLGVIWSGGSIDYNAAFISRLSISKDNSKSQVFIKMNSLQADDTAIYYCARGPRKFYYFGMDYWGQGTSVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 35 - protein, 470 aa, Artificial Sequence
  Chimeric RF2 VH and canine HCD constant domain
MEWSWVFLFFLSVTTGVHSQVQLKQSGPGLVPPSQSLSITCTISGFSLTSYGVHWVRQSPGWGLEWLGVIWSGGSIDYNAAFISRLSISKDNSKSQVFIKMNSLQADDTAIYYCARGPRKFYYFGMDYWGQGTSVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFYPPEIDVEWQSNGQPEPESKYHTTAPQLDEDGSYFLYSKLSVDKSRWQQGDTFTCAVMHEALQNHYTDLSLSHSPGK

SEQ ID NO: 36 - protein, 242 aa, Artificial Sequence
  Chimeric RF2 VL and canine kLC constant domain
MSVPTQVLGLLLLWLTDARCDVVMTQTPLSLPVSLGDQASISCRSSQNIVHSNGNTYVEWYLQKPGQSPKLLIYKVSNRFSGVPDRFSGSGSGTDFTLKISRVEAEDLGVYYCFQGSHVPLTFGAGTKLELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 37 - protein, 120 aa, Artificial Sequence
  Caninised RA2 Heavy chain VH
EVQLVQSGGGLVKPTESLTISCVVSGFSLTNYGVHWVRQSPGKGLEWLGVIWSGGTTDYNAAFISRLSISKDNSKSTVFLRMNSLRADDTAIYYCARGPRKFYYYGMDYWGQGTSVTVSS

SEQ ID NO: 38 - protein, 112 aa, Artificial Sequence
  Caninised RA2 Light chain VL
DIVMTQTPLSLSVSQEEEASISCRSSQSIVHSNGNTYLEWYLQKPGQSPKLLIYKVSNRFSGVPDRFSGSGSGTDFTLKISRVEAGDAGVYYCFQGLHVPLTFGAGTKLELK

SEQ ID NO: 39 - protein, 470 aa, Artificial Sequence
  Complete caninised RA2 heavy chain HCA
MEWSWVFLFFLSVTTGVHSEVQLVQSGGGLVKPTESLTISCVVSGFSLTNYGVHWVRQSPGKGLEWLGVIWSGGTTDYNAAFISRLSISKDNSKSTVFLRMNSLRADDTAIYYCARGPRKFYYYGMDYWGQGTSVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 40 - protein, 474 aa, Artificial Sequence
  Complete caninised RA2 heavy chain HCB
MEWSWVFLFFLSVTTGVHSEVQLVQSGGGLVKPTESLTISCVVSGFSLTNYGVHWVRQSPGKGLEWLGVIWSGGTTDYNAAFISRLSISKDNSKSTVFLRMNSLRADDTAIYYCARGPRKFYYYGMDYWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 41 - protein, 472 aa, Artificial Sequence
  Complete caninised RA2 heavy chain HCC
MEWSWVFLFFLSVTTGVHSEVQLVQSGGGLVKPTESLTISCVVSGFSLTNYGVHWVRQSPGKGLEWLGVIWSGGTTDYNAAFISRLSISKDNSKSTVFLRMNSLRADDTAIYYCARGPRKFYYYGMDYWGQGTSVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 42 - protein, 470 aa, Artificial Sequence
  Complete caninised RA2 heavy chain HCD
MEWSWVFLFFLSVTTGVHSEVQLVQSGGGLVKPTESLTISCVVSGFSLTNYGVHWVRQSPGKGLEWLGVIWSGGTTDYNAAFISRLSISKDNSKSTVFLRMNSLRADDTAIYYCARGPRKFYYYGMDYWGQGTSVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 43 - protein, 242 aa, Artificial Sequence
  Complete caninised RA2 light chain kLC
MSVPTQVLGLLLLWLTDARCDIVMTQTPLSLSVSQEEEASISCRSSQSIVHSNGNTYLEWYLQKPGQSPKLLIYKVSNRFSGVPDRFSGSGSGTDFTLKISRVEAGDAGVYYCFQGLHVPLTFGAGTKLELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 44 - protein, 120 aa, Artificial Sequence
  Caninised RF2 heavy chain VH
EVQLVQSGGGLVKPAESLTISCVISGFSLTSYGVHWVRQSPGKGLEWLGVIWSGGSIDYNAAFISRLSISKDNSKSTVFIRMNSLRADDTAIYYCARGPRKFYYFGMDYWGQGTSVTVSS

SEQ ID NO: 45 - protein, 112 aa, Artificial Sequence
  Caninised RF2 light chain VL
DIVMTQTPLSLSVSQEEEASISCRSSQNIVHSNGNTYVEWYLQKPGQSPKLLIYKVSNRFSGVPDRFSGSGSGTDFTLKISRVEAEDAGVYYCFQGSHVPLTFGAGTKLELK

SEQ ID NO: 46 - protein, 470 aa, Artificial Sequence
  Complete caninised RF2 heavy chain HCA
MEWSWVFLFFLSVTTGVHSEVQLVQSGGGLVKPTESLTISCVISGFSLTSYGVHWVRQSPGKGLEWLGVIWSGGSIDYNAAFISRLSISKDNSKSTVFIRMNSLRADDTAIYYCARGPRKFYYFGMDYWGQGTSVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 47 - protein, 474 aa, Artificial Sequence
  Complete caninised RF2 heavy chain HCB
MEWSWVFLFFLSVTTGVHSEVQLVQSGGGLVKPTESLTISCVISGFSLTSYGVHWVRQSPGKGLEWLGVIWSGGSIDYNAAFISRLSISKDNSKSTVFIRMNSLRADDTAIYYCARGPRKFYYFGMDYWGQGTSVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 48 - protein, 472 aa, Artificial Sequence
  Complete caninised RF2 heavy chain HCC
MEWSWVFLFFLSVTTGVHSEVQLVQSGGGLVKPTESLTISCVISGFSLTSYGVHWVRQSPGKGLEWLGVIWSGGSIDYNAAFISRLSISKDNSKSTVFIRMNSLRADDTAIYYCARGPRKFYYFGMDYWGQGTSVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 49 - protein, 470 aa, Artificial Sequence
  Complete caninised RF2 heavy chain HCD
MEWSWVFLFFLSVTTGVHSEVQLVQSGGGLVKPTESLTISCVISGFSLTSYGVHWVRQSPGKGLEWLGVIWSGGSIDYNAAFISRLSISKDNSKSTVFIRMNSLRADDTAIYYCARGPRKFYYFGMDYWGQGTSVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 50 - protein, 242 aa, Artificial Sequence
  Complete caninised RF2 light chain kLC
MSVPTQVLGLLLLWLTDARCDIVMTQTPLSLSVSQEEEASISCRSSQNIVHSNGNTYVEWYLQKPGQSPKLLIYKVSNRFSGVPDRFSGSGSGTDFTLKISRVEAEDAGVYYCFQGSHVPLTFGAGTKLELKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 51 - protein, 120 aa, Artificial Sequence
  Felinised  RA2 heavy chain VH
QVQLVQSGGELVTPGESLSITCVVSGFSLTNYGVHWVRQSPGKGLEWVGVIWSGGTTDYNAAFISRLTISKDNSKNTVFLQMNSLQADDTAIYYCARGPRKFYYYGMDYWGQGTSVTVSS

SEQ ID NO: 52 - protein, 112 aa, Artificial Sequence
  Felinised RA2 light chain VL
DVVMTQTPLSLPVTPGDSASISCRSSQSIVHSNGNTYLEWYLQKPGQSPRLLIYKVSNRFSGVPDRFSGSGSGTDFTLKISRVEADDLGVYYCFQGLHVPLTFGQGTKLELK

SEQ ID NO: 53 - protein, 120 aa, Artificial Sequence
  Felinised RF2 heavy chain VH
QVQLVQSGGELVTPGESLSITCVVSGFSLTSYGVHWVRQSPGKGLEWVGVIWSGGSIDYNAAFISRLTISKDNSKNTVFLQMNSLQADDTAIYYCARGPRKFYYFGMDYWGQGTSVTVSS

SEQ ID NO: 54 - protein, 112 aa, Artificial Sequence
  Felinised RF2 light chain VL
DVVMTQTPLSLPVTPGDSASISCRSSQNIVHSNGNTYVEWYLQKPGQSPRLLIYKVSNRFSGVPDRFSGSGSGTDFTLKISRVEAEDLGVYYCFQGSHVPLTFGQGTKLELK

SEQ ID NO: 55 - protein, 8 aa, Artificial Sequence
  RA2 CDRH1
SLTNYGVH

SEQ ID NO: 56 - protein, 16 aa, Artificial Sequence
  RA2 CDRH2
VIWSGGTTDYNAAFIS

SEQ ID NO: 57 - protein, 12 aa, Artificial Sequence
  RA2 CDRH3
GPRKFYYYGMDY

SEQ ID NO: 58 - protein, 16 aa, Artificial Sequence
  RA2 CDRL1
RSSQSIVHSNGNTYLE

SEQ ID NO: 59 - protein, 7 aa, Artificial Sequence
  RA2 CDRL2
KVSNRFS

SEQ ID NO: 60 - protein, 9 aa, Artificial Sequence
  RA2 CDRL3
FQGLHVPLT

SEQ ID NO: 61 - protein, 8 aa, Artificial Sequence
  RF2 CDRH1
SLTSYGVH

SEQ ID NO: 62 - protein, 16 aa, Artificial Sequence
  RF2 CDRH2
VIWSGGSIDYNAAFIS

SEQ ID NO: 63 - protein, 12 aa, Artificial Sequence
  RF2 CDRH3
GPRKFYYFGMDY

SEQ ID NO: 64 - protein, 16 aa, Artificial Sequence
  RF2 CDRL1
RSSQNIVHSNGNTYVE

SEQ ID NO: 65 - protein, 7 aa, Artificial Sequence
  RF2 CDRL2
KVSNRFS

SEQ ID NO: 66 - protein, 9 aa, Artificial Sequence
  RF2 CDRL3
FQGSHVPLT

SEQ ID NO: 67 - protein, 5 aa, Canis familiaris
SEKNS

SEQ ID NO: 68 - protein, 6 aa, Canis familiaris
SEKNSL

SEQ ID NO: 69 - protein, 6 aa, Canis familiaris
PSEKNS

(19) United States
(12) Patent Application Publication
(10) Pub. No.: US 2014/0294819 A1
(43) Pub. Date: Oct. 2, 2014

(54) CANINE/FELINE CD20 BINDING EPITOPE AND COMPOSITIONS FOR BINDING THERETO

(71) Applicant: NVIP Pty Ltd, Melbourne, Victoria (AU)

(72) Inventor: David Gearing, Melbourne (AU)

(21) Appl. No.: 14/351,392

(22) PCT Filed: Oct. 12, 2012
(86) PCT No.: PCT/GB2012/052532

§ 371 Date: Apr. 11, 2014

Related U.S. Application Data
(60) Provisional application No. 61/546,865, filed on Oct. 13, 2011.

(51) Int. Cl. C07K 16/28; C12P 21/00; G01N 33/68; C07K 7/64

(52) U.S. Cl. CPC C07K 16/2887; C07K 7/64; C12P 21/00; G01N 33/6872
USPC 424/133.1; 530/387.9; 424/139.1; 530/387.3; 536/23.53; 530/317; 514/21.1; 424/185.1; 435/320.1; 435/331; 435/69.6; 435/7.92

(57) ABSTRACT

The present invention provides acyclic polypeptide fragment of CD20 comprising (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide is oxidised by the presence of a disulphide bond formed between the first and second cysteine residues. Also described is the use of the cyclic peptide fragment to generate antibodies which bind specifically to CD20. Antibodies which bind specifically to the cyclic peptide fragment for use in treatment of B-cell mediated conditions in felines and canines are also described.

### FIELD OF THE INVENTION

The present invention relates to the identification of a binding epitope which is bound by antibodies which have binding specificity to CD20. The invention extends to the use of this epitope to generate antibodies and to the use of antibodies which bind thereto in the treatment and diagnosis of disease conditions, such as lymphoma and immune mediated diseases, in canines and felines. The invention further extends to antibodies which bind to the identified epitope.

### BACKGROUND TO THE INVENTION

CD20 is an activated glycosylated phosphoprotein expressed on the surface of most B-cells, beginning at the pro-B phase and progressively increasing in concentration until maturity. The protein has no known natural ligand and its function is to enable an optimal B-cell immune response, specifically against T-cell independent antigens. It functions as a calcium channel in the cell membrane. It is involved in intracellular signal transduction and can also modulate cell growth and differentiation.

CD20 is an established marker or target for B-cell lymphomas due to the expression of this antigen at high levels on malignant B cells which are associated with B cell lymphoma. Targeting of CD20 (which is also known as Bp35) using monoclonal antibodies has been proven to be a successful therapeutic approach in the treatment of B-cell lymphomas in humans, and also in the treatment of immune-mediated conditions, such as rheumatoid arthritis. Human CD20 is the target of the monoclonal antibodies rituximab, Ibritumomab tiuxetan and tositumomab, which are all active agents in the treatment of B-cell lymphomas.

Companion animals such as dogs and cats develop similar diseases to humans, including lymphoma, immune-mediated polyarthritidies, plasmatic-lymphocytic synovitis, systemic lupus erythematosus, vasculitis and a variety of autoimmune skin diseases. Canine lymphoma is the second most prevalent cancer in dogs whereas lymphoma is the most common malignancy diagnosed in cats. With current combination therapy, the expected survival time for dogs with B-cell lymphoma is around 9 to 12 months. Side effects of the standard chemotherapeutic regimes are similar to those seen in humans and include vomiting, diarrhea, lack of appetite, fever and sepsis.

Canine CD20 has been characterised and predicted to contain two extracellular (EC) domains, four transmembrane (TM) domains, and three intracellular (IC) domains as in human CD20. While canine CD20 has structural homology with human CD20, anti-human and anti-murine CD20 monoclonal antibodies are reportedly incapable of binding to canine CD20. The identification of antibodies which have binding specificity to canine and/or feline CD20 could have particular utility in the treatment or diagnosis of canines and felines with B-cell lymphoma.

### SUMMARY OF THE INVENTION

Following extensive experimentation, the present inventor has surprisingly identified a novel antigenic loop of the canine and feline CD20 polypeptide which is capable of being specifically bound by anti-human CD20 monoclonal antibodies when said polypeptide loop sequence is constrained by a disulphide bond provided between first and second cysteine amino acid residues. As a result, the inventor has identified for the first time that certain monoclonal antibodies which have binding specificity to human CD20 can also have therapeutic and diagnostic applications in canines and felines, due to those antibodies having binding specificity for canine and/or feline CD20. Specifically, the identified novel epitope may have particular utility in the identification of novel anti-canine or anti-feline CD20 antibodies for use in the diagnosis, treatment and/or prophylaxis of CD20+ B-cell lymphoma and immune mediated conditions.

According to a first aspect of the invention, there is provided an antibody or an antigen binding fragment thereof that specifically binds to a cyclic polypeptide fragment of CD20 for use in the treatment or prevention of a condition mediated by B-cells in a canine or feline subject in need thereof, wherein the cyclic polypeptide fragment comprises, consists of or consists essentially of (i) a contiguous amino acid sequence comprising, consisting of or consisting essentially of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide fragment is oxidised by the presence of a disulphide bond formed between the first and second cysteine residues.

According to a second aspect of the present invention there is provided a method for treating or preventing a condition mediated by B-cells in a canine or feline subject in need thereof, the method comprising the step of administering a therapeutically effective amount of an antibody or an antigen binding fragment thereof that specifically binds to a cyclic polypeptide fragment of CD20, wherein the cyclic polypeptide fragment comprises (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide fragment is oxidised by the presence of a disulphide bond formed between the first and second cysteine residues.

In certain embodiments the method of this aspect of the invention can further comprise the step of administering at least one immunosuppressive compound to the canine or feline subject. The immunosuppressive compound may be administered before, along with (simultaneously) or after (sequentially) the administration of the antibody or antigen binding fragment. The immunosuppressive agent may be selected from the group consisting of a growth factor inhibitor, an immunosuppressor such as an antibody, an anti-inflammatory, an enzymatic inhibitor, a steroid, a non-steroid anti-inflammatory drug, a metabolic inhibitor, a cytotoxic agent and a cytostatic agent.

In embodiments wherein the condition mediated by B cells is rheumatoid arthritis, the antibody can optionally be administered in conjunction with a second therapeutic agent, which is preferably methotrexate.

According to a third aspect of the present invention there is provided use of an antibody or an antigen binding fragment thereof that specifically binds to a cyclic polypeptide fragment of CD20 in the preparation of a medicament for the treatment or prevention of a condition mediated by B-cells in a canine or feline subject in need thereof, wherein the cyclic polypeptide fragment comprises (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide fragment is oxidised by the presence of a disulphide bond formed between the first and second cysteine residues.

The present inventor has identified that the cyclic polypeptide fragment of the above aspects of the invention forms an epitope. The epitope formed by the cyclic polypeptide fragment is common to humans, canines, felines and mice. In certain embodiments the contiguous amino acid sequence comprises, consists of or consists essentially of amino acid residues SEKNSL (SEQ ID NO:68). The present inventor has identified that the epitope formed by the cyclic polypeptide fragment comprising SEQ ID NO:68 is common to canines and felines. In certain embodiments the contiguous amino acid sequence comprises, consists of or consists essentially of amino acid residues PSEKNS (SEQ ID NO:69). The present inventor has identified that the epitope formed by the cyclic polypeptide fragment comprising SEQ ID NO:69 is common to humans, canines and felines. In certain embodiments the contiguous amino acid sequence comprises, consists of or consists essentially of amino acid residues PSEKNSL (SEQ ID NO 1). The present inventor has identified that the epitope formed by the cyclic polypeptide fragment comprising SEQ ID NO:1 is common to canines and felines.

In certain embodiments the cyclic polypeptide fragment comprises, consists of or consists essentially of SEQ ID NO:2 or an amino acid sequence having at least 85%, 90% or 95% sequence identity thereto. Typically the subject is a canine.

In certain embodiments the cyclic polypeptide fragment comprises, consists of or consists essentially of SEQ ID NO:4 or an amino acid sequence having at least 85%, 90% or 95% sequence identity thereto. Typically the subject is a feline.

In certain embodiments the antibody is derived from a Type II anti-human or anti-murine CD20 antibody, for example, B1-H299, GA101 or Bly1. In certain embodiments the antibody is a caninised or felinised antibody, for example, a caninised or felinised Type II anti-human or anti-murine CD20 antibody such as caninised or felinised B1-H299, GA101 or Bly1 antibody.

In certain embodiments the antibody or antigen binding fragment thereof is derived from GA101. Typically where the subject is a canine, the antibody or antigen binding fragment may comprise a light chain variable region comprising at least one of an FR1 framework region consisting of or comprising the amino acid sequence of SEQ ID NO:7, an FR2 framework region consisting of or comprising the amino acid sequence of SEQ ID NO:8, an FR3 framework region consisting of or comprising the amino acid sequence of SEQ ID NO:9, and an FR4 framework region consisting of or comprising the amino acid sequence of SEQ ID NO:10, and/or a heavy chain variable region comprising at least one of an FR1 framework region consisting of or comprising the amino acid sequence of SEQ ID NO:11, an FR2 framework region consisting of or comprising the amino acid sequence of SEQ ID NO:12, an FR3 framework region consisting of or comprising the amino acid sequence of SEQ ID NO:13, and an FR4 framework region consisting of or comprising the amino acid sequence of SEQ ID NO:14. In certain embodiments the light chain variable region comprises all of the aforementioned light chain framework regions and/or the heavy chain variable region comprises all of the aforementioned heavy chain framework regions. In certain embodiments the antibody or antigen binding fragment thereof comprises a light chain variable region comprising the amino acid sequence of SEQ ID NO:15 or an amino acid sequence which has an identity of at least 85%, 90%, 95% or 98% thereto and/or a heavy chain variable region comprising the amino acid sequence of SEQ ID NO:16 or an amino acid sequence which has an identity of at least 85%, 90%, 95% or 98% thereto. In certain embodiments the antibody or antigen binding fragment thereof comprises a light chain comprising the amino acid sequence of SEQ ID NO:18 or an amino acid sequence which has an identity of at least 85%, 90%, 95% or 98% thereto and/or a heavy chain variable region comprising the amino acid sequence of SEQ ID NO:17 or an amino acid sequence which has an identity of at least 85%, 90%, 95% or 98% thereto.

In certain embodiments the antibody or antigen binding fragment thereof comprises a heavy chain variable region comprising a complementarity determining region 1 (CDR1) region comprising the amino acid sequence of SEQ ID NO:55, a CDR2 region comprising the amino acid sequence of SEQ ID NO:56 and a CDR3 region comprising the amino acid sequence of SEQ ID NO:57, a light chain variable region comprising a CDR1 region comprising the amino acid sequence of SEQ ID NO:58, a CDR2 region comprising the amino acid sequence of SEQ ID NO:59 and a CDR3 region comprising the amino acid sequence of SEQ ID NO:60. An antibody having the above CDRs is termed RA2.

In certain embodiments the antibody or antigen binding fragment thereof is derived from RA2, for example, the antibody or antigen binding fragment may be caninised or felinised RA2.

In certain embodiments wherein the subject is a canine and the RA2 is caninised RA2 the heavy chain variable region comprises the amino acid sequence of SEQ ID NO:37 or an amino acid sequence which has an identity of at least 85%, 90%, 95% or 98% thereto and/or the light chain variable region comprises the amino acid sequence of SEQ ID NO:38 or an amino acid sequence which has an identity of at least 85%, 90%, 95% or 98% thereto. In certain embodiments wherein the subject is a canine the heavy chain comprises an amino acid sequence selected from the group consisting of SEQ ID NO:39, SEQ ID NO:40, SEQ ID NO:41 and SEQ ID NO:42, or an amino acid sequence which has an identity of at least 85%, 90%, 95% or 98% to SEQ ID NO:39, SEQ ID NO:40, SEQ ID NO:41 or SEQ ID NO:42. In certain embodiments wherein the subject is a canine the light chain comprises the amino acid sequence of SEQ ID NO:43 or an amino acid sequence which has an identity of at least 85%, 90%, 95% or 98% thereto.

In certain embodiments wherein the subject is a feline and the RA2 is felinised the heavy chain variable region comprises the amino acid sequence of SEQ ID NO:51 or an amino acid sequence which has an identity of at least 85%, 90%, 95% or 98% thereto and/or the light chain variable region comprises the amino acid sequence of SEQ ID NO:52 or an amino acid sequence which has an identity of at least 85%, 90%, 95% or 98% thereto.

In certain embodiments the antibody or antigen binding fragment comprises a chimeric antibody, for example, comprising a constant domain of a canine or feline heavy and/or light chain. In certain embodiments wherein the subject is a canine the antibody or antigen binding fragment comprises chimeric RA2, for example, the heavy chain comprises an amino acid sequence selected from the group consisting of SEQ ID NO:27, SEQ ID NO:28, SEQ ID NO:29 and SEQ ID NO:30, or an amino acid sequence which has an identity of at least 85%, 90%, 95% or 98% to SEQ ID NO:27, SEQ ID NO:28, SEQ ID NO:29 or SEQ ID NO:30. In certain embodiments wherein the subject is a canine the light chain comprises the amino acid sequence of SEQ ID NO:31 or an amino acid sequence which has an identity of at least 85%, 90%, 95% or 98% thereto.

In certain embodiments the antibody or antigen binding fragment thereof comprises a heavy chain variable region comprising a complementarity determining region 1 (CDR1) region comprising the amino acid sequence of SEQ ID NO:61, a CDR2 region comprising the amino acid sequence of SEQ ID NO:62 and a CDR3 region comprising the amino acid sequence of SEQ ID NO:63, a light chain variable region comprising a CDR1 region comprising the amino acid sequence of SEQ ID NO:64, a CDR2 region comprising the amino acid sequence of SEQ ID NO:65 and a CDR3 region comprising the amino acid sequence of SEQ ID NO:66. An antibody having the above CDRs is termed RF2.

In certain embodiments the antibody or antigen binding fragment thereof is derived from RF2, for example, the antibody or antigen binding fragment may be caninised or felinised RF2.

In certain embodiments wherein the subject is a canine and the antibody is caninised RF2 the heavy chain variable region comprises the amino acid sequence of SEQ ID NO:44 or an amino acid sequence which has an identity of at least 85%, 90%, 95% or 98% thereto and/or the light chain variable region comprises the amino acid sequence of SEQ ID NO:45 or an amino acid sequence which has an identity of at least 85%, 90%, 95% or 98% thereto. In certain embodiments wherein the subject is a canine the heavy chain comprises an amino acid sequence selected from the group consisting of SEQ ID NO:46, SEQ ID NO:47, SEQ ID NO:48 and SEQ ID NO:49, or an amino acid sequence which has an identity of at least 85%, 90%, 95% or 98% to SEQ ID NO:46, SEQ ID NO:47, SEQ ID NO:48 or SEQ ID NO:49. In certain embodiments wherein the subject is a canine the light chain comprises the amino acid sequence of SEQ ID NO:50 or an amino acid sequence which has an identity of at least 85%, 90%, 95% or 98% thereto.

In certain embodiments wherein the subject is a feline and the antibody is felinised RF2 the heavy chain variable region comprises the amino acid sequence of SEQ ID NO:53 or an amino acid sequence which has an identity of at least 85%, 90%, 95% or 98% thereto and/or the light chain variable region comprises the amino acid sequence of SEQ ID NO:54 or an amino acid sequence which has an identity of at least 85%, 90%, 95% or 98% thereto.

In certain embodiments wherein the subject is a canine the antibody or antigen binding fragment comprises chimeric RF2, for example, the heavy chain comprises an amino acid sequence selected from the group consisting of SEQ ID NO:32, SEQ ID NO:33, SEQ ID NO:34 and SEQ ID NO:35, or an amino acid sequence which has an identity of at least 85%, 90%, 95% or 98% to SEQ ID NO:32, SEQ ID NO:33, SEQ ID NO:34 or SEQ ID NO:35. In certain embodiments wherein the subject is a canine the light chain comprises the amino acid sequence of SEQ ID NO:36 or an amino acid sequence which has an identity of at least 85%, 90%, 95% or 98% thereto.

In certain embodiments the B-cell mediated condition is a hematologic malignancy characterised by a high number of tumour cells in the blood or a B-cell lymphoma, in particular a CD20+ B-cell lymphoma. In certain embodiments the condition is a hematologic malignancy which is characterised by lower B-cell CD20 expression levels, such as conditions selected from the group consisting of transformed non-Hodgkin's lymphoma, precursor B-cell lymphoblastic leukemia/lymphoma and mature B-cell neoplasms, such as B-cell chronic lymphocytic leukemia (CLL)/small lymphocytic lymphoma (SLL), B-pro-lymphocytic leukaemia (B-PLL), lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma, Burkitt's lymphoma, plasmacytoma, plasma cell myeloma, post-transplant lymphoproliferative disorder, Waldenstrom's macroglobulinemia and anaplastic large-cell lymphoma (ALCL). In certain embodiments the condition is non-Hodgkin's lymphoma, such as relapsed and previously treated low-grade non-Hodgkin's lymphoma (NHL).

In certain embodiments the condition mediated by B-cells is an immune mediated disease. Typically the immune mediated disease is an autoimmune disease, an immune disorder or an inflammatory disease and may be selected from the group consisting of rheumatoid arthritis, systemic lupus erythematosus (SLE), Sjogren's syndrome, vasculitis, multiple sclerosis, Graves' disease, idiopathic thrombocytopenia, dermatomyositis, immune mediated thrombocytopenia, polymyocytosis, pemphigus, immune mediated haemolytic anaemia and bullous pemphigoid. In certain embodiments the immune mediated disease is selected from the group consisting of juvenile rheumatoid arthritis, Wegener's disease, inflammatory bowel disease, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Crohn's disease, ulcerative colitis, gastritis, Hashimoto's thyroiditis, ankylosing spondylitis, hepatitis C-associated cryoglobulinemic vasculitis, chronic focal encephalitis, bullous pemphigoid, hemophilia A, membranoproliferative glomerulnephritis, adult and juvenile dermatomyositis, polymyositis, chronic uticaria, primary biliary cirrhosis, neuromyelitis optica, Graves' dysthyroid disease, membranoproliferative glonerulonephritis, Churg-Strauss syndrome, asthma, psoriatic arthritis, dermatitis, respiratory distress syndrome, meningitis, encephalitits, uveitis, eczema, atherosclerosis, leukocyte adhesion deficiency, juvenile onset diabetes, Reiter's disease, Behcet's disease, hemolytic anemia, atopic dermatitis, pemphigus vulgaris, Wegener's granulomatosis, Omenn's syndrome, chronic renal failure, acute infectious mononucleosis, HIV and herpes-associated disease, systemic sclerosis and glomerulonephritis. In the foregoing conditions, it is understood that depleting B-cells may provide a therapeutic approach to treating such condition.

In certain embodiments, a chemical agent or radioactive label may be conjugated to the antibody or antigen binding fragment in order that the chemical agent or label is specifically delivered to neoplastic B-cells. Said chemical agent or radioactive label has the potential to destroy CD20 expressing cells.

According to a fourth aspect of the present invention there is provided a caninised or felinised antibody or an antigen binding fragment thereof which binds specifically to a cyclic polypeptide fragment of CD20, wherein the cyclic polypeptide fragment comprises, consists of or consists essentially of (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide fragment is oxidised by the presence of a disulphide bond formed between the first and second cysteine residues.

In certain embodiments the antibody is a caninised antibody comprising complementarity determining regions of a heavy and/or light chain from a donor antibody from a species other than a canine, wherein the donor antibody has binding specificity for the cyclic polypeptide fragment. In certain embodiments the antibody comprises framework regions of the heavy and/or light chain from the donor antibody. In certain embodiments the framework regions of the heavy and/or light chain from the donor antibody are modified to substitute amino acid residues that are foreign at a corresponding position in canine antibodies with amino acid residues present at the corresponding position in canine antibodies.

In certain embodiments the antibody is a felinised antibody comprising complementarity determining regions of a heavy and/or light chain from a donor antibody from a species other than a feline, wherein the donor antibody has binding specificity for the cyclic polypeptide fragment. In certain embodiments the antibody comprises framework regions of the heavy and/or light chain from the donor antibody. In certain embodiments the framework regions of the heavy and/or light chain from the donor antibody are modified to substitute amino acid residues that are foreign at a corresponding position in feline antibodies with amino acid residues present at the corresponding position in feline antibodies.

Typically the amino acid residues that are foreign at the corresponding position in canine or feline antibodies are substituted with the amino acid residues present at the corresponding position which have the highest homology to the substituted amino acid residues.

Typically the antibody or antigen binding fragment comprises constant domains of a heavy and/or light chain from a canine or feline antibody.

In certain embodiments the antibody is derived from (that is, a caninised or felinised version of) a Type II anti-human or anti-murine CD20 antibody, for example, the antibody may be selected from the group consisting of B1-H299, GA101 and Bly1.

In certain embodiments the antibody is derived from (that is, a caninised or felinised version of) RA2 or RF2, for example, as described above.

According to a fifth aspect of the present invention there is provided a humanised antibody or an antigen binding fragment thereof which binds specifically to a cyclic polypeptide fragment of CD20, wherein the cyclic polypeptide fragment comprises (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide fragment is oxidised by the presence of a disulphide bond formed between the first and second cysteine residues, and wherein framework regions of the heavy and/or light chain are derived from an antibody obtained from a species other than human and the framework regions are modified to substitute amino acid residues that are foreign at a corresponding position inhuman antibodies with amino acid residues present at the corresponding position in human antibodies.

In certain embodiments the amino acid residues that are foreign at the corresponding position inhuman antibodies are substituted with the amino acid residues present at the corresponding position which have the highest homology to the one or more substituted amino acid residues.

Typically the antibody or antigen binding fragment comprises constant domains of a heavy and/or light chain from a human antibody.

According to a sixth aspect of the present invention there is provided a chimeric antibody or an antigen binding fragment thereof which binds specifically to a cyclic polypeptide fragment of CD20, wherein the cyclic polypeptide fragment comprises (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide fragment is oxidised by the presence of a disulphide bond formed between the first and second cysteine residues and wherein the antibody comprises a canine or feline constant domain.

According to a seventh aspect of the present invention there is provided an antibody or an antigen binding fragment thereof, which specifically binds to CD20 wherein the antibody or fragment thereof comprises a heavy chain variable region comprising a complementarity determining region 1 (CDR1) region comprising the amino acid sequence of SEQ ID NO:55, a CDR2 region comprising the amino acid sequence of SEQ ID NO:56 and a CDR3 region comprising the amino acid sequence of SEQ ID NO:57, a light chain variable region comprising a CDR1 region comprising the amino acid sequence of SEQ ID NO:58, a CDR2 region comprising the amino acid sequence of SEQ ID NO:59 and a CDR3 region comprising the amino acid sequence of SEQ ID NO:60.

In certain embodiments the heavy chain variable region comprises the amino acid sequence of SEQ ID NO:19 or an amino acid sequence which has an identity of at least 85%, 90%, 95% or 98% thereto and/or the light chain variable region comprises the amino acid sequence of SEQ ID NO:20 or an amino acid sequence which has an identity of at least 85%, 90%, 95% or 98% thereto. In certain embodiments the heavy chain comprises the amino acid sequence of SEQ ID NO:23 or an amino acid sequence which has an identity of at least 85%, 90%, 95% or 98% thereto and/or the light chain comprises the amino acid sequence of SEQ ID NO:24 or an amino acid sequence which has an identity of at least 85%, 90%, 95% or 98% thereto.

In certain embodiments the antibody is a caninised antibody. Typically the heavy chain variable region comprises the amino acid sequence of SEQ ID NO:37 or an amino acid sequence which has an identity of at least 85%, 90%, 95% or 98% thereto and/or the light chain variable region comprises the amino acid sequence of SEQ ID NO:38 or an amino acid sequence which has an identity of at least 85%, 90%, 95% or 98% thereto. In certain embodiments the heavy chain comprises an amino acid sequence selected from the group consisting of SEQ ID NO:39, SEQ ID NO:40, SEQ ID NO:41 and SEQ ID NO:42, or an amino acid sequence which has an identity of at least 85%, 90%, 95% or 98% to SEQ ID NO:39, SEQ ID NO:40, SEQ ID NO:41 or SEQ ID NO:42. In certain embodiments the light chain comprises the amino acid sequence of SEQ ID NO:43 or an amino acid sequence which has an identity of at least 85%, 90%, 95% or 98% thereto.

In certain embodiments the antibody is a felinised antibody. Typically the heavy chain variable region comprises the amino acid sequence of SEQ ID NO:51 or an amino acid sequence which has an identity of at least 85%, 90%, 95% or 98% thereto and/or the light chain variable region comprises the amino acid sequence of SEQ ID NO:52 or an amino acid sequence which has an identity of at least 85%, 90%, 95% or 98% thereto.

In certain embodiments the antibody is a humanised antibody.

In certain embodiments the antibody is a chimeric antibody. Typically the heavy chain and/or light chain comprises a constant domain derived from a canine, feline or human antibody. In certain embodiments the heavy chain comprises an amino acid sequence selected from the group consisting of SEQ ID NO:27, SEQ ID NO:28, SEQ ID NO:29 and SEQ ID NO:30, or an amino acid sequence which has an identity of at least 85%, 90%, 95% or 98% to SEQ ID NO:27, SEQ ID NO:28, SEQ ID NO:29 or SEQ ID NO:30. In certain embodiments the light chain comprises the amino acid sequence of SEQ ID NO:31 or an amino acid sequence which has an identity of at least 85%, 90%, 95% or 98% thereto.

Typically the antibody or antigen binding fragment is cross-reactive and specifically binds to human, murine, canine and feline CD20.

According to an eighth aspect of the present invention there is provided an antibody or an antigen binding fragment thereof, which specifically binds to CD20 wherein the antibody or fragment thereof comprises a heavy chain variable region comprising a complementarity determining region 1 (CDR1) region comprising the amino acid sequence of SEQ ID NO:61, a CDR2 region comprising the amino acid sequence of SEQ ID NO:62 and a CDR3 region comprising the amino acid sequence of SEQ ID NO:63, a light chain variable region comprising a CDR1 region comprising the amino acid sequence of SEQ ID NO:64, a CDR2 region comprising the amino acid sequence of SEQ ID NO:65 and a CDR3 region comprising the amino acid sequence of SEQ ID NO:66.

In certain embodiments the heavy chain variable region comprises the amino acid sequence of SEQ ID NO:21 or an amino acid sequence which has an identity of at least 85%, 90%, 95% or 98% thereto and/or the light chain variable region comprises the amino acid sequence of SEQ ID NO:22 or an amino acid sequence which has an identity of at least 85%, 90%, 95% or 98% thereto. In certain embodiments the heavy chain comprises the amino acid sequence of SEQ ID NO:25 or an amino acid sequence which has an identity of at least 85%, 90%, 95% or 98% thereto and/or the light chain comprises the amino acid sequence of SEQ ID NO:26 or an amino acid sequence which has an identity of at least 85%, 90%, 95% or 98% thereto.

In certain embodiments the antibody is a caninised antibody. Typically the heavy chain variable region comprises the amino acid sequence of SEQ ID NO:44 or an amino acid sequence which has an identity of at least 85%, 90%, 95% or 98% thereto and/or the light chain variable region comprises the amino acid sequence of SEQ ID NO:45 or an amino acid sequence which has an identity of at least 85%, 90%, 95% or 98% thereto. In certain embodiments the heavy chain comprises an amino acid sequence selected from the group consisting of SEQ ID NO:46, SEQ ID NO:47, SEQ ID NO:48 and SEQ ID NO:49, or an amino acid sequence which has an identity of at least 85%, 90%, 95% or 98% to SEQ ID NO:46, SEQ ID NO:47, SEQ ID NO:48 or SEQ ID NO:49. In certain embodiments the light chain comprises the amino acid sequence of SEQ ID NO:50 or an amino acid sequence which has an identity of at least 85%, 90%, 95% or 98% thereto.

In certain embodiments the antibody is a felinised antibody. Typically the heavy chain variable region comprises the amino acid sequence of SEQ ID NO:53 or an amino acid sequence which has an identity of at least 85%, 90%, 95% or 98% thereto and/or the light chain variable region comprises the amino acid sequence of SEQ ID NO:54 or an amino acid sequence which has an identity of at least 85%, 90%, 95% or 98% thereto.

In certain embodiments the antibody is a humanised antibody.

In certain embodiments the antibody is a chimeric antibody. Typically the heavy chain and/or light chain comprises a constant domain derived from a canine, feline or human antibody. In certain embodiments the heavy chain comprises an amino acid sequence selected from the group consisting of SEQ ID NO:32, SEQ ID NO:33, SEQ ID NO:34 and SEQ ID NO:35, or an amino acid sequence which has an identity of at least 85%, 90%, 95% or 98% to SEQ ID NO:32, SEQ ID NO:33, SEQ ID NO:34 or SEQ ID NO:35. In certain embodiments the light chain comprises the amino acid sequence of SEQ ID NO:36 or an amino acid sequence which has an identity of at least 85%, 90%, 95% or 98% thereto.

Typically the antibody or antigen binding fragment is cross-reactive and specifically binds to human, murine, canine and feline CD20.

According to a ninth aspect of the present invention there is provided an antibody or an antigen binding fragment thereof comprising a heavy chain variable region comprising the amino acid sequence of SEQ ID NO:16 or an amino acid sequence which has an identity of at least 85%, 90%, 95% or 98% thereto and/or a light chain variable region comprising the amino acid sequence of SEQ ID NO:15 or an amino acid sequence which has an identity of at least 85%, 90%, 95% or 98% thereto.

In certain embodiments the heavy chain comprises the amino acid sequence of SEQ ID NO:17 or an amino acid sequence which has an identity of at least 85%, 90%, 95% or 98% thereto and/or the light chain comprises the amino acid sequence of SEQ ID NO:18 or an amino acid sequence which has an identity of at least 85%, 90%, 95% or 98% thereto.

In certain embodiments of the fourth to ninth aspects of the invention the antibody or antigen binding fragment thereof specifically binds to canine and/or feline CD20 with a binding affinity having an equilibrium dissociation constant (K_{D}) of 1×10⁻⁸ or less.

Typically the antibody or antigen binding fragment is an isolated antibody or antigen binding fragment thereof.

In certain embodiments the antibody or antigen binding fragment is selected from the group consisting of a single chain Fv (scFv) antibody fragment, a Fab antibody fragment, a Fab′ antibody fragment and a F(ab′)₂ antibody fragment. In certain embodiments the antibody or antigen binding fragment is a multispecific or multivalent antibody.

In certain embodiments, a chemical agent may be conjugated to an antibody or antigen binding fragment according to any one of the fourth to ninth aspects in order that the chemical agent is specifically delivered to neoplastic B-cells. Said chemical agent or radioactive label has the potential to destroy CD20 expressing cells. Accordingly, the invention extends to immunoconjugates that consist of cytotoxic agents conjoined to an antibody or fragment thereof by means of a chemical linker, said immunoconjugates also being known as antibody-cytotoxic agent conjugates (ACC) or antibody drug conjugates (ADC). Such immunoconjugates allow the targeted delivery of the drug moiety to tumour cells.

Examples of drugs which are useful in this regard include methotrexate and vindesine, while toxins include, but are not limited to, bacterial toxins, plant toxins such as ricin and small molecule toxins such as geldanamycin.

In certain embodiments, a radioactive label may be conjugated to an antibody or antigen binding fragment according to any one of the fourth to ninth aspects in order that the radioactive label is specifically delivered to neoplastic B-cells. In certain embodiments the label may be selected from the group comprising, but not limited to, a radiolabel, a fluorophore, a chromophore, an imaging agent and a metal ion. Typically the labelled antibody or fragment may have utility in diagnosis.

The invention therefore further provides a method for diagnosing a subject suspected of having a condition mediated by B-cells, said method comprising administering to a subject an antibody or antigen binding fragment conjugated to a label and detecting the distribution of the antibody or antigen binding fragment within the subject. In various embodiments the method of diagnosis includes diagnosing B-cell mediated disorder, immune disorder, autoimmune disease or inflammatory disease selected from the list defined hereinbefore.

According to a tenth aspect of the present invention there is provided an isolated nucleic acid that encodes an antibody or antigen binding fragment according to any one of the fourth to ninth aspects of the invention.

Also provided is an expression vector comprising said nucleic acid and a host cell incorporating the expression vector. The invention further extends to a method for producing an antibody comprising the step of culturing said host cell to allow the cell to express the antibody.

The invention further extends to an antibody or antigen binding fragment according to any one of the fourth to ninth aspects of the invention for use in the treatment or prevention of a condition mediated by B-cells.

Also provided is a method for treating or preventing a condition mediated by B-cells comprising the steps of administering a therapeutically effective amount of an antibody or antigen binding fragment according to any one of the fourth to ninth aspects of the invention to a subject in need thereof.

The invention also extends to use of an antibody or antigen binding fragment according to any one of the fourth to ninth aspects of the invention in the preparation of a medicament for the treatment or prevention of a condition mediated by B-cells.

In certain embodiments the condition mediated by B-cells is any condition as described above in relation to the first to third aspects of the invention.

Also provided is an antibody or antigen binding fragment according to any one of the fourth to ninth aspects of the invention for use in diagnosis.

The invention further provides a cyclic polypeptide fragment of CD20 comprising, consisting or consisting essentially of (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide is oxidised by the presence of a disulphide bond formed between the first and second cysteine residues.

In certain embodiments the contiguous amino acid sequence comprises, consists of or consists essentially of amino acid residues SEKNSL (SEQ ID NO:68). In certain embodiments the contiguous amino acid sequence comprises, consists of or consists essentially of amino acid residues PSEKNS (SEQ ID NO:69). In certain embodiments the contiguous amino acid sequence comprises, consists of or consists essentially of amino acid residues PSEKNSL (SEQ ID NO 1).

In certain embodiments the cyclic polypeptide fragment comprises less than 30, 28, 25, 24, 23, 22 or 21 amino acid residues.

In certain embodiments the cyclic polypeptide fragment comprises, consists of or consists essentially of SEQ ID NO:2 or an amino acid sequence having at least 85%, 90% or 95% sequence identity thereto.

In certain embodiments the cyclic polypeptide fragment comprises, consists of or consists essentially of SEQ ID NO:4 or an amino acid sequence having at least 85%, 90% or 95% sequence identity thereto.

In certain embodiments the cyclic polypeptide fragment comprises, consists of or consists essentially of SEQ ID NO:3 or an amino acid sequence having at least 85%, 90% or 95% sequence identity thereto.

In certain embodiments the cyclic polypeptide fragment comprises, consists of or consists essentially of SEQ ID NO:6 or an amino acid sequence having at least 85%, 90% or 95% sequence identity thereto.

Typically binding of the cyclic polypeptide fragment by an antagonistic binding member antagonises CD20 biological activity.

The invention further extends to a pharmaceutical composition comprising the cyclic polypeptide fragment and a pharmaceutically acceptable carrier or excipient.

The invention further extends to a vaccine composition comprising the cyclic polypeptide fragment. In certain embodiments the cyclic polypeptide fragment is connected to or otherwise associated with one or more amino acid sequences comprising T-cell epitopes recognisable by the target species of the vaccine. Typical examples of such amino acid sequences include tetanus toxoid or fragments thereof, diphtheria toxoid or fragments thereof and keyhole limpet haemocyanin (KLH). In certain embodiments the vaccine will further comprise an adjuvant selected to increase the magnitude of the immune response elicited to the vaccine. Typical examples of adjuvants include aluminium salts, oil-in-water and water-in-oil emulsions, saponins and Lipid A and its derivatives and homologues.

Also provided is use of the cyclic polypeptide fragment, such as a cyclic polypeptide fragment of SEQ ID NO:2, in a method for generating a binding member which specifically binds to CD20.

In certain embodiments the method is a method for generating a binding member which specifically binds to canine CD20. In certain embodiments the method is a method for generating a binding member which specifically binds to feline CD20.

The invention further provides a method for generating a binding member which specifically binds to CD20, the method comprising the steps of:
administering to a subject a cyclic polypeptide fragment as described above, such as a cyclic polypeptide fragment of SEQ ID NO:2, and
isolating binding agents which bind specifically to said cyclic polypeptide fragment.

In certain embodiments the method is a method for generating a binding member which specifically binds to canine CD20. In certain embodiments the method is a method for generating a binding member which specifically binds to feline CD20.

The invention further provides a screening method for identifying a binding member which specifically binds to canine and/or feline CD20, the screening method comprising the steps of:
bringing a candidate compound into contact with a cyclic polypeptide fragment as described above, such as the cyclic polypeptide fragment comprising SEQ ID NO:2; and
assessing binding between the candidate compound and the cyclic polypeptide fragment;
   wherein binding between the candidate compound and the cyclic polypeptide fragment identifies the candidate compound as a binding member which specifically binds to canine and/or feline CD20.

In certain embodiments the candidate compound is selected from the group consisting of proteins, such as antibodies, peptides, such as fusion protein, peptidomimetics, nucleic acids, polynucleotides, polysaccharides, oligopeptides, carbohydrates, lipids, small molecule compounds and naturally occurring compounds.

The invention further provides a method for detecting the presence of canine or feline CD20 in a B-lymphocyte-containing sample comprising the steps of:
contacting one or more antibodies as described above with the sample under conditions that allow B-lymphocyte/antibody complexes to form; and
detecting B-lymphocyte/antibody complexes, wherein the detection of said complexes is an indication that canine or feline CD20 is present in the sample.

In certain embodiments, the method comprises the further step of determining whether the subject from which the sample was obtained has B-cell lymphoma by means of detecting B-lymphocyte/antibody complexes, wherein the presence of said complexes provides an indication that the subject from which the sample originated has a B-cell mediated condition, such as B-cell lymphoma.

A yet further aspect of the invention provides a cell line, or a derivative or progeny cell thereof that produces an antibody or an antigen binding fragment thereof according to any foregoing aspect of the invention.

A further still aspect provides a kit for the treatment or diagnosis of CD20+ B cell lymphoma in a canine comprising an anti-canine CD20 antibody according to any foregoing aspect of the invention. A further still aspect provides a kit for the treatment or diagnosis of CD20+ B cell lymphoma in a feline comprising an anti-feline CD20 antibody according to any foregoing aspect of the invention.

A yet further aspect provides a kit for the treatment of an immune mediated condition, comprising an anti-canine CD20 antibody according to any foregoing aspect of the invention. A yet further aspect provides a kit for the treatment of an immune mediated condition, comprising an anti-feline CD20 antibody according to any foregoing aspect of the invention.

### BRIEF DESCRIPTION OF THE DRAWINGS

FIG. 1 shows the amino acid sequence of the canine CD20 derived cyclic polypeptide fragment having the amino acid sequence of SEQ ID NO:2, with the disulphide bond between the two cysteine residues corresponding to positions 167 and 183 shown.

FIGS. 2A and 2B show the binding of H299 and Biogenex B1 (BG) monoclonal antibodies to disulphide bonded human (H20), murine (M20) and canine (C20) CD20 polypeptide fragments.

FIG. 3 shows the binding of H299 monoclonal antibody to both human and canine CD20 expressed on CHO cells. FIG. 3A shows the binding of H299 monoclonal antibody (6 μg/ml) to canine CD20 CHO cells. FIG. 3B shows the binding of H299 monoclonal antibody (6 μg/ml) to human CD20 CHO cells. FIG. 3C shows the binding of secondary anti-mouse PE antibody (Sigma P9287) to canine CD20 CHO cells. FIG. 3D shows the binding of secondary anti-mouse PE antibody (Sigma P9287) to human CD20 CHO cells.

FIG. 4 shows a comparison of binding of two different Type II anti-human CD20 monoclonal antibodies (H299 and Bly1) to a disulphide bonded canine CD20 polypeptide fragment.

FIG. 5 shows the binding by FACS of two different Type II anti-human CD20 monoclonal antibodies (H299 and Bly1) to human CD20- and canine CD20-expressing CHO cells.

FIG. 6 shows that the binding of the Type II monoclonal antibody H299 to a disulphide-bonded canine CD20 peptide is abolished by reduction of the disulphide bond with dithiothreitol (DTT), whereas the binding to disulphide bonded human CD20 peptide is reduced, but not abolished, by DTT treatment.

FIG. 7 shows the complete caninised GA101 VK kappa variable light chain amino sequence (FIG. 7a—SEQ ID NO:15) and variable heavy chain (FIG. 7b—SEQ ID NO:16) wherein the CDR region residues are shown in bold.

FIG. 8 shows recombinant caninised GA101 (humanised Bly1) constructed from co-expressed SEQ ID NO:17 and 18 binds to canine CD20 peptide SEQ ID NO:2.

FIG. 9 shows binding of novel murine MAbs RA2 and RF2 to cyclised canine, human, mouse and feline CD20 peptides—human CD20 cyclic peptide (SEQ ID NO:3) (RA2 weakly) and canine CD20 cyclised peptide (SEQ ID NO:2) as well as murine (SEQ ID NO:6) and feline (SEQ ID NO:4) CD20 peptides.

FIG. 10 a to d show the derived variable domain heavy and light chain sequences of RA2 (a,b) and RF2 (c,d) monoclonal antibodies wherein CDRs are underlined. FIG. 10e shows an alignment of RA2 and RF2 variable domains.

FIG. 11 shows binding of recombinant mouse RA2 and RF2 MAbs to cyclised CD20 peptides.

FIG. 12 shows an alignment of peptide loops from canine, feline, human and mouse CD20 and illustrates the shorter common peptide epitope SEKNS, a sub-epitope of PSEKNSL deduced from the common binding of Type II anti-human CD20 monoclonal antibodies to human and canine CD20.

FIG. 13 shows the heavy (FIG. 13a) and light chain (FIG. 13b) variable domains of caninised RA2.

FIG. 14 shows the heavy (FIG. 14a) and light (FIG. 14b) chain variable domains of caninised RF2.

FIG. 15 shows SDS-PAGE analysis of CHO cell expressed and Protein A purified forms of murine, murine/canine chimeric and caninised RA2 and RF2 antibodies.

FIG. 16 shows the binding by ELISA of recombinant chimeric and caninised RA2 and RF2 monoclonal antibodies to canine, feline, mouse and human cyclised CD20 peptides.

FIG. 17 shows binding of complement C1q to caCD20 peptide-immobilised chimeric and caninised RA2 and RF2.

FIG. 18 shows binding of soluble recombinant canine high affinity Fc receptor (scaCD64) to caCD20 peptide-immobilised chimeric and caninised RA2 and RF2.

### DETAILED DESCRIPTION OF THE INVENTION

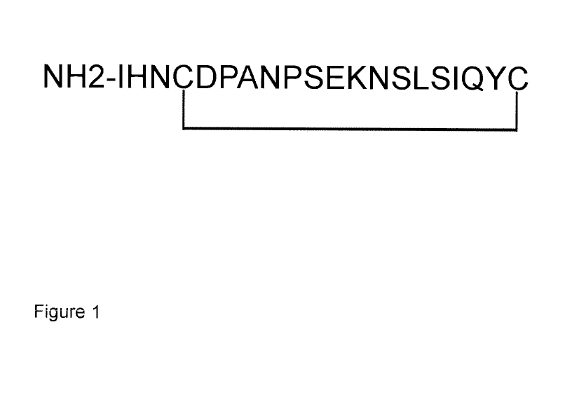

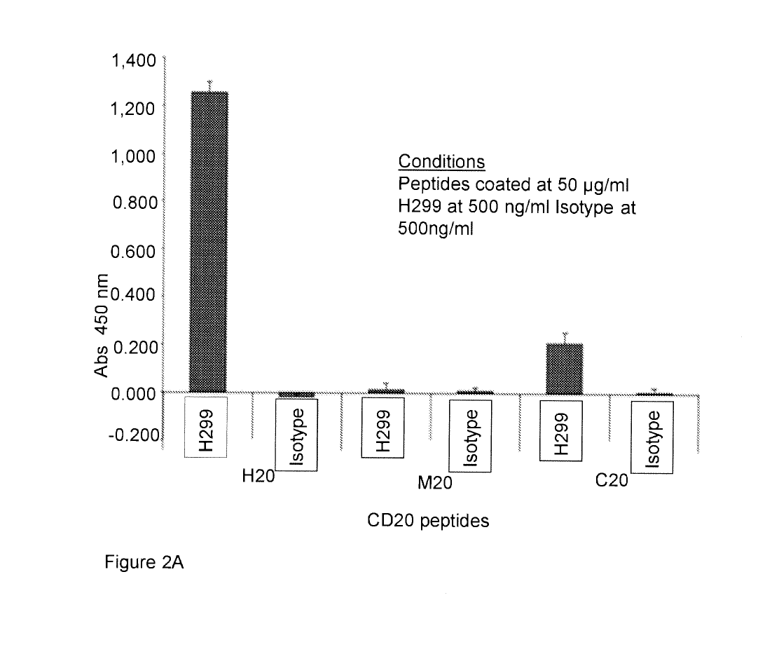

The present invention relates to the identification of a disulphide-bonded and structurally-constrained antigenic loop of canine CD20 that binds specifically to Type II anti-human CD20 monoclonal antibodies. The amino acid sequence of this novel epitope of CD20 is shown in FIG. 1, where the disulphide bond between the two cysteine residues is shown. The defined epitope, when bound by a binding agent which binds specifically thereto, results in antagonism of the function of CD20.

Without wishing to be bound by theory, the inventor has surprisingly identified that anti-human and/or anti-murine CD20 monoclonal antibodies can bind to a novel disulphide-bonded and structurally-constrained antigenic loop of canine CD20. In particular, the inventor has shown that the binding of monoclonal antibodies to canine CD20 is critically dependent on the conformation of the polypeptide, with the conformation being dependent on the presence of a disulphide bond provided between two cysteine residues.

In particular, the inventor has identified a polypeptide which is derived from canine CD20 which comprises the amino acid sequence of SEQ ID NO:2. In addition, following the appreciation that the presence of cysteine residues are necessary for the presence of a disulphide bond, the inventor has observed that cysteine residues which are used to form the disulphide bond are conserved across CD20 proteins of several species. In particular, homologous disulphide bonded peptides from CD20 derived from different species may be useful in identifying diagnostic and therapeutic binding agents for use in the diagnosis and therapy of CD20+ B cell mediated conditions. Such species include human, feline, equine and murine having the polypeptide sequences set out in SEQ ID NO:3; SEQ ID NO:4; SEQ ID NO:5 and SEQ ID NO:6 respectively.

Anti-CD20 monoclonal antibodies can be classified as Type I and Type II anti-CD20 monoclonal antibodies. This classification is dependent on the mechanism of action used to kill malignant B-cells. Type I (rituximab-like) monoclonal antibodies induce CD20 to redistribute into large lipid rafts or microdomains in the plasma membrane, whereas Type II (tositumomab-like) do not. Importantly, this redistribution of CD20 impacts many of the binding properties and effector functions that control the therapeutic success of anti-CD20 monoclonal antibodies.

The inventor has surprisingly found that the Type II anti-human CD20 monoclonal antibodies H299 and GA101 (including its parent mouse monoclonal antibody Bly1) bind to not only human CD20, but also canine CD20 and that the polypeptide of SEQ ID NO:2 allows this determination to be made. The present inventor has also surprisingly shown that other Type II anti-human CD20 monoclonal antibodies bind to canine CD20. This is surprising as Type I anti-human CD20 monoclonal antibodies, such as Rituxan, do not bind canine CD20.

According to Polyak and Deans (Blood 99, 3256; 2002) the Type II anti-human CD20 monoclonal antibodies H299 and Bly1 bind to a similar epitope on human CD20. Niederfellner et al. (2011) determined that H299 and Bly1 bind to human CD20 with dependence on each of contiguous residues 172-PSEKNSP-178. Type I anti-human CD20 monoclonal antibodies, such as Rituximab and C2H7 have binding which is dependent on the more N-terminal contiguous residues 168-EPANPSEK-175. These residues are aligned with canine and murine CD20 peptides below (Type I underlined, Type II in italics):

Human
(SEQ ID NO: 3)
IYNCEPANPSEKNSPSTQYC
Canine
(SEQ ID NO: 2)
IHNCDPANPSEKNSLSIQYC
Murine
(SEQ ID NO: 6)
IYDCEPSNSSEKNSPSTQYC

Further investigation by the inventor has surprisingly identified an analogous sequence in felines:

Feline
(SEQ ID NO: 4)
IHTCQPESKPSEKNSLSIKYC

The present invention therefore further relates to the identification of a disulphide-bonded and structurally-constrained antigenic loop of feline CD20.

Without wishing to be bound by theory, the inventor has identified that the lack of binding of Rituximab to canine CD20 is due to canine sequence amino acid residue D168 (E in the human sequence). Furthermore, the inventor has also identified that a critical region for binding Type II anti-human CD20 monoclonal antibodies such as H299 monoclonal antibody in human CD20 is the P178 residue, which is L in canine CD20. The inventor concludes that it is the P178L amino acid difference between human and canine CD20 polypeptides of SEQ ID NO:2 and SEQ ID NO:3 which explains why H299 binds more weakly to canine CD20 than it does to human CD20.

The inventor has taken the GA101 anti-human CD20 antibody which was not known to bind to canine CD20 and produced antibodies which bind specifically to canine CD20. Furthermore, the inventor has provided RA2 and RF2 anti-murine CD20 antibodies which bind specifically to human, canine, murine and feline CD20. The caninised and felinised and humanised antibodies of the invention, which are not produced using standard CDR grafting techniques, are shown to exhibit high affinity binding to canine, human and/or feline CD20. The antibodies have been designed so that the framework and constant regions incorporate only residues present in canine, human or feline IgG molecules as appropriate so that when administered to a canine, human or feline, xeno antibodies are unlikely to be produced there against.

The process of generating the heavy and light chain variable domains for the antibodies of the invention which has been employed by the inventor results in the replacement of specific framework donor amino acid residues known to be foreign to the target species at that position with a residue which is found at that position in the target species (e.g. canine, feline or human) and which, based on the inventor's analysis, will retain the conformation of the CDR regions and therefore maintain binding specificity and avidity, while reducing the presence of immunogenic epitopes which may result in neutralising antibodies being generated against the antibody if it were to be administered to target species in an unaltered form. Specifically, the method of preparing antibodies of the invention (known as PETisation) comprises assessing the sequence of the framework regions of a donor (e.g. human) antibody for suitability for administering to a target species by comparing the sequence of the framework regions of the donor antibody with the sequence of an antibody or a pool of antibodies derived from the target species. Although the comparison may be between the donor sequence and a single member of the target sequence, it will be obvious that comparison with a pool of target sequences is preferred because this will expand the number of natural options at each Kabat position in the target species. Not only will this increase the chance of a “match” between the donor and the target, but it will also expand the options for replacement where a match does not exist. As a result, a replacement with characteristics as close as possible to the donor will be able to be chosen. Where the donor sequence and the target sequence differ at any Kabat number or corresponding position, the donor sequence is modified to substitute the amino acid residue in question with an amino acid residue which is known to be natural at that position in the target species.

Where substitution of an amino acid residue present in a donor immunoglobulin framework region is required, typically this is undertaken using the principle of conservative substitution wherein an amino acid residue is replaced with an amino acid residue which is natural at that Kabat position in a target species and is as closely related as possible in size, charge and hydrophobicity to the amino acid being substituted in the donor sequence. The intention is to choose a replacement which would cause no, or at least only minimum, perturbation or disruption to the three-dimensional structure of the donor antibody. In certain situations, there will be no clear option and each choice will have benefits and downsides. A final decision may require three-dimensional modelling or even expression of various alternative sequences. However, generally, a clear preference will be available. As a result of this procedure, a change in the donor sequence is only made when that residue would be foreign in the target and the replacement amino acid is as closely related as possible to that which it replaces. Thus, the creation of foreign epitopes is avoided, but the overall three-dimensional structure is preserved and as a result, affinity and specificity are also preserved.

The term “epitope” as used herein relates to a portion or portions of a macromolecule which is capable of being bound by a specific antibody, in this case, a portion of a polypeptide, in particular CD20. Epitopes generally consist of chemically active surface groups and have specific three dimensional structural characteristics, as well as specific charge characteristics. Typically, the CD20 binding agent or binding compound antagonises the binding activity of CD20 and as such binds to an epitope known as an inhibiting epitope or an inhibitory epitope. An “inhibiting” or “inhibitory” epitope means an epitope present on CD20, that when bound by a binding compound such as a small molecule or an antibody, results in the loss of biological activity of CD20.

Epitopes may be defined from contiguous or non-contiguous sequences of amino acid residues comprised within a polypeptide sequence. The term “contiguous epitope” defines an epitope comprised of a linear series of amino acid residues within a polypeptide which define the epitope. A contiguous epitope may be conformational if the peptide is conformationally constrained e.g. by forming a loop. A “non-contiguous epitope”, which may also be referred to as a conformational and discontinuous epitope, is an epitope which is comprised of a series of amino acid residues which are non-linear in alignment, that is that the residues are spaced or grouped in a non-continuous manner along the length of a polypeptide sequence. A non-contiguous epitope can be a discontinuous epitope wherein the amino acid residues are grouped into 2 linear sequences, or alternatively the non-contiguous epitope can be a discontinuous scattered epitope wherein the residues which contribute to the epitope are provided in 3 or more groups of linear amino acid sequences arranged along the length of the polypeptide.

As herein defined an “antibody” encompasses antigen-binding proteins which specifically bind to a target antigen of interest, in this case canine and/or feline CD20 including polypeptides that can be recombinantly prepared or which are genetically encodable by immunoglobulin genes, or fragments of immunoglobulin genes. The term “antibody” encompasses monoclonal and chimeric antibodies, in particular caninised and felinised antibodies, and further encompasses polyclonal antibodies or antibodies of any class or subtype. An “antibody” further extends to hybrid antibodies, bispecific antibodies, hetero antibodies and to functional fragments thereof which retain antigen binding.

The constant region of the antibody may be of any suitable immunoglobulin subtype, however it is preferred that the antibody subtype is IgG. Such an antibody may further belong to any subclass e.g. in the canine, IgG-A, IgG-B, IgG-C and IgG-D and in certain embodiments be either of the subclass IgG-B or IgG-C. However, in certain embodiments, the subtype of the antibody may be of the class IgA, IgM, IgD or IgE.

Fragments of a whole antibody can perform the function of antigen binding. Examples of such binding fragments are a Fab fragment comprising the VL, VH, CL and CH1 antibody domains; an Fv fragment consisting of the VL and VH domains of a single antibody; a F(ab′)2 fragment, a bivalent fragment comprising two linked Fab fragments; a single chain Fv molecule (scFv), wherein a VH domain and a VL domain are linked by a peptide linker which allows the two domains to associate to form an antigen binding site; or a bi-specific antibody, which may be multivalent or multispecific fragments constructed by gene fusion.

A fragment of an antibody or of a polypeptide for use in the present invention, for example, a fragment of a polypeptide defining a canine CD20 binding epitope or an antibody which binds specifically to CD20 and in particular to CD20 at the epitope defined by SEQ ID NO:2, generally means a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids. Similarly, a fragment of a polypeptide defining a feline CD20 binding epitope or an antibody which binds specifically to CD20 and in particular to CD20 at the epitope defined by SEQ ID NO:4, generally means a stretch of amino acid residues of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

A “derivative” of such an antibody or polypeptide, or of a fragment of a CD20 specific antibody means an antibody or polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, preferably while providing a peptide having CD20 binding activity. Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

The term “derived from” as used herein to refer to an antibody or antigen binding fragment being derived from a specific antibody is understood to mean that the antibody or antigen binding fragment may be a chimeric or modified (e.g. caninised or felinised) version of the specific antibody. Typically the antibody or antigen binding fragment which is derived from the specific antibody will retain the CDRs of the specific antibody, but may comprise different constant and/or framework regions.

In certain embodiments the antibody is an “isolated antibody”. This is understood to mean that the antibody is (1) free of at least some proteins with which it would normally be found, (2) is essentially free of other proteins from the same source, e.g., from the same species, (3) is expressed by a cell from a different species, or (4) does not occur in nature.

The term “caninised” as used herein is understood to mean that the antibody has been modified for use in a canine, for example, by substituting one or more amino acids which would be foreign at a corresponding position in a canine with amino acids found at the corresponding position in a canine. The term “felinised” as used herein is understood to mean that the antibody has been modified for use in a feline, for example, by substituting one or more amino acids which would be foreign at a corresponding position in a feline with amino acids found at the corresponding position in a feline. The term “humanised” as used herein is understood to mean that the antibody has been modified for use in a human, for example, by substituting one or more amino acids which would be foreign at a corresponding position in a human with amino acids found at the corresponding position in a human.

Amino acids which are “foreign” in a target species may be identified by comparing an amino acid sequence of, for example, a framework region of a donor antibody with amino acid sequence(s) of, for example, a framework region of one or more antibodies from the target species to identify one or more amino acid residues in the donor framework region which are not present at the corresponding position in antibodies from the target species. The target species may be canine, feline or human.

The term “corresponding position” as used herein to refer to an amino acid residue that is present in a second sequence at a position corresponding to a specified amino acid residue in a first sequence is intended to refer to the position in the second sequence which is the same position as the position in the first sequence when the two sequences are aligned to allow for maximum sequence identity between the two sequences. Amino acid residues at corresponding positions have the same Kabat numbering.

The phrase “specifically binds to” refers to the binding of an antibody to a specific protein or target which is present amongst a heterogeneous population of proteins. Hence, when present in specific immunoassay conditions, the antibodies bind to a particular protein, in this case canine or feline CD20 and in particular to the epitope defined by SEQ ID NO:2 of 4, and do not bind in a significant amount to other proteins present in the sample.

The term “subject” as used herein may refer to a mammal, e.g. a canine, feline or human. Typically the subject may be suffering from a B-cell mediated condition as described above.

The phrase “at a region N-terminal to” as used herein is understood to mean that the first cysteine residue may be present directly adjacent the N-terminus of the contiguous amino acid sequence or, more typically, one or more amino acid residues may separate the first cysteine residue from the N-terminus of the contiguous amino acid sequence. Similarly, the phrase “at a region C-terminal to” as used herein is understood to mean that the second cysteine residue may be present directly adjacent the C-terminus of the contiguous amino acid sequence or, more typically, one or more amino acid residues may separate the second cysteine residue from the C-terminus of the contiguous amino acid sequence.

The variable region other than the hypervariable region may also be derived from the variable region of a human antibody and/or may also be derived from a monoclonal antibody, such as a CD20 specific antibody. In such case, the entire variable region may be derived from a murine monoclonal antibody, such as a CD20 specific antibody and the antibody is said to be chimerised. Methods for making chimerised antibodies are known in the art.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. A hybridoma or other cell producing an antibody may be subject to genetic mutation or other changes, which may or may not alter the binding specificity of antibodies produced.

### Production of Antibodies

Certain methodologies for producing antibodies which have an affinity and binding specificity for the CD20 epitopes of the present invention are described hereinbefore.

The antibodies or antibody fragments of and for use in the present invention may also be generated wholly or partly by chemical synthesis. The antibodies can be readily prepared according to well-established, standard liquid or, preferably, solid-phase peptide synthesis methods, general descriptions of which are broadly available and are well known by the person skilled in the art. Further, they may be prepared in solution, by the liquid phase method or by any combination of solid-phase, liquid phase and solution chemistry.

Another convenient way of producing antibodies or antibody fragments suitable for use in the present invention is to express nucleic acid encoding them by use of nucleic acid in an expression system.

Nucleic acid for use in accordance with the present invention may comprise DNA or RNA and may be wholly or partially synthetic. Nucleic acid for use in the invention may code for antibodies or antibody fragments of the invention as defined above. The skilled person will be able to determine substitutions, deletions and/or additions to such nucleic acids which will still provide an antibody or antibody fragment of the present invention.

Nucleic acid sequences encoding antibodies or antibody fragments for use with the present invention can be readily prepared by the skilled person using the information and references contained herein and techniques known in the art given the nucleic acid sequences and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding antibody fragments may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the sequences can be made, e.g. using site directed mutagenesis, to lead to the expression of modified peptide or to take account of codon preferences in the host cells used to express the nucleic acid.

The nucleic acid may be comprised as constructs in the form of a plasmid, vector, transcription or expression cassette which comprises at least one nucleic acid as described above. The construct may be comprised within a recombinant host cell which comprises one or more constructs as above. Expression may conveniently be achieved by culturing under appropriate conditions recombinant host cells containing the nucleic acid. Following production by expression the antibody or antibody fragments may be isolated and/or purified using any suitable technique, then used as appropriate.

Systems for cloning and expression of a polypeptide in a variety of different host cells are well known. Suitable host cells include bacteria, mammalian cells, yeast, insect and baculovirus systems. Mammalian cell lines available in the art for expression of a heterologous polypeptide include Chinese hamster ovary (CHO) cells, HeLa cells, baby hamster kidney cells, NS0 mouse myeloma cells. A common, preferred bacterial host is E. coli. The expression of antibodies and antibody fragments in prokaryotic cells such as E. coli is well established in the art. Expression in eukaryotic cells in culture is also available to those skilled in the art as an option for production of a binding member.

Recombinant nucleic acids comprising an insert coding for a heavy chain variable domain and/or for a light chain variable domain of antibodies may be employed. By definition such nucleic acids comprise coding single stranded nucleic acids, double stranded nucleic acids consisting of said coding nucleic acids and of complementary nucleic acids thereto, or these complementary (single stranded) nucleic acids themselves. Furthermore, nucleic acids encoding a heavy chain variable domain and/or a light chain variable domain of antibodies can be enzymatically or chemically synthesised nucleic acids having the authentic sequence coding for a naturally-occurring heavy chain variable domain and/or for the light chain variable domain, or a mutant thereof.

Recombinant DNA technology may be used to improve the antibodies of the invention. Thus, chimeric antibodies may be constructed in order to decrease the immunogenicity thereof in diagnostic or therapeutic applications. In order to reduce immunogenicity within a recipient, the invention may employ recombinant nucleic acids comprising an insert coding for a heavy chain variable domain of an antibody fused to a canine or feline constant domain. Likewise the invention concerns recombinant DNAs comprising an insert coding for a light chain variable domain of an antibody fused to a canine constant domain κ or λ.

Antibodies may moreover be generated by mutagenesis of antibody genes to produce artificial repertoires of antibodies. This technique allows the preparation of antibody libraries. Antibody libraries are also available commercially. Hence, the present invention advantageously employs artificial repertoires of immunoglobulins, preferably artificial scFv repertoires, as an immunoglobulin source in order to identify binding molecules which have specificity for the epitope of the present invention.

### Antibody Selection Systems

Immunoglobulins which are able to bind to the epitope of the present invention and which accordingly may be used in the methods of the invention can be identified using any technique known to the skilled person. Such immunoglobulins may be conveniently isolated from libraries comprising artificial repertoires of immunoglobulin polypeptides. A “repertoire” refers to a set of molecules generated by random, semi-random or directed variation of one or more template molecules, at the nucleic acid level, in order to provide a multiplicity of binding specificities. Methods for generating repertoires are well characterised in the art.

Any library selection system may be used in conjunction with the invention. Selection protocols for isolating desired members of large libraries are known in the art, as typified by phage display techniques. Such systems, in which diverse peptide sequences are displayed on the surface of filamentous bacteriophage, have proven useful for creating libraries of antibody fragments (and the nucleotide sequences that encode them) for the in vitro selection and amplification of specific antibody fragments that bind a target antigen. The nucleotide sequences encoding the VH and VL regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage capsids (phage bodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straight forward. Methods for the construction of bacteriophage antibody display libraries and lambda phage expression libraries are well known in the art.

An alternative to the use of phage or other cloned libraries is to use nucleic acid, preferably RNA, derived from the B cells of an animal which has been immunised with the selected target, e.g. the CD20 epitope of the present invention. Isolation of V-region and C-region mRNA permits antibody fragments, such as Fab or Fv, to be expressed intracellularly. Briefly, RNA is isolated from the B cells of an immunised animal, for example from the spleen of an immunised mouse or the circulating B cells of a llama, and PCR primers used to amplify VH and VL cDNA selectively from the RNA pool. The VH and VL sequences thus obtained are joined to make scFv antibodies. PCR primer sequences may be based on published VH and VL sequences.

### Peptidomimetics

Peptide analogues, such as peptidomimetics or peptide mimetics are non-peptide compounds with properties representative of a template peptide. Such peptide analogues are typically developed using computerised molecular modelling. Peptidomimetics which are structurally similar to peptides which have affinity and binding specificity to the CD20 binding epitope of the present invention may be used to mediate similar diagnostic, prophylactic and therapeutic effects.

Peptidomimetics are typically structurally similar to a template peptide, but have one or more peptide linkages replaced by an alternative linkage, by methods which are well known in the art. A peptide may further be modified from the natural sequence to protect the peptides from protease attack.

### Sequence Homology/Identity

The disulphide-bonded epitope of the present invention comprises an amino acid sequence with a disulphide bond between two cysteine residues as defined in SEQ ID NO:2, SEQ ID NO:3, SEQ ID NO:4, SEQ ID NO:5 or SEQ ID NO:6.

The term “identity” or “sequence identity” as used herein, means that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. This may be referred to as conservative substitution. Preferably when the amino acid sequences of the invention are modified by way of conservative substitution of any of the amino acid residues contained therein, these changes have no effect on the binding specificity or functional activity of the resulting antibody when compared to the unmodified antibody.

Sequence identity with respect to a (native) polypeptide of the invention and its functional derivative relates to the percentage of amino acid residues in the candidate sequence which are identical with the residues of the corresponding native polypeptide, after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology. Methods and computer programs for performing an alignment of two or more amino acid sequences and determining their sequence identity or homology are well known to the person skilled in the art. For example, the percentage of identity or similarity of 2 amino acid sequences can be readily calculated using algorithms e.g. BLAST (Altschul et al. 1990), FASTA (Pearson & Lipman 1988), or the Smith-Waterman algorithm (Smith & Waterman 1981).

The term “consists essentially of” or “consisting essentially of” as used herein means that a polypeptide may have additional features or elements beyond those described provided that such additional features or elements do not materially affect the ability of an antibody or antibody fragment to bind to the epitope defined by the polypeptide. That is, the polypeptides may have additional features or elements that do not interfere with their ability to present an epitope which can be bound by binding agents which are specific for canine and/or feline CD20. For example, a polypeptide consisting essentially of a specified sequence may contain one, two, three, four, five or more additional, deleted or substituted amino acids, at either end or at both ends of the sequence provided that these amino acids do not interfere with, inhibit, block or interrupt the role of the antibody or fragment in binding to canine or feline CD20. Similarly, a polypeptide of the invention may be chemically modified with one or more functional groups provided that such functional groups do not interfere with the function of the polypeptide.

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide.

The invention extends to the use of the peptide which has been determined as contributing to the epitope in binding to CD20 ligands. As such, the invention extends to polypeptide fragments of the amino acid of SEQ ID NO:2, SEQ ID NO:3, SEQ ID NO:4, SEQ ID NO:5 or SEQ ID NO:6 of varying lengths, wherein the fragments define a binding epitope according to the present invention, which when bound by a ligand with a specific binding affinity, results in an inhibition of CD20 mediated signalling.

The epitope may be provided in an isolated form in order to assist in the production of antibodies and binding fragments which have affinity and binding specificity to the identified binding epitope. Accordingly, the present invention extends to naturally occurring fragments and variants as well as derived variants of a polypeptide having the amino acid sequence of SEQ ID NO:2, SEQ ID NO:3, SEQ ID NO:4, SEQ ID NO:5 or SEQ ID NO:6.

A “variant” of a polypeptide having the amino acid sequence of SEQ ID NO:2, SEQ ID NO:3, SEQ ID NO:4, SEQ ID NO:5 or SEQ ID NO:6 means a polypeptide substantially homologous to a polypeptide having the amino acid sequence of SEQ ID NO:2, SEQ ID NO:3, SEQ ID NO:4, SEQ ID NO:5 or SEQ ID NO:6, but which has an amino acid sequence different from that of the polypeptide having the amino acid sequence of SEQ ID NO:2, SEQ ID NO:3, SEQ ID NO:4, SEQ ID NO:5 or SEQ ID NO:6 because of one or more deletions, insertions, or substitutions. The variant has an amino acid sequence that preferably is at least 80% identical to the polypeptide having the amino acid sequence of SEQ ID NO:2, SEQ ID NO:3, SEQ ID NO:4, SEQ ID NO:5 or SEQ ID NO:6, most preferably at least 90% identical. The percentage identity may be determined, for example, by comparing sequence information using the GAP computer program, version 6.0 described by Devereux et al. (Nucl. Acids Res. 12:387, 1984) and available from the University of Wisconsin Genetics Computer Group (UWGCG).

The present invention extends to peptides which are variants, derivates or homologues of a polypeptide having the amino acid sequence of SEQ ID NO:2, SEQ ID NO:3, SEQ ID NO:4, SEQ ID NO:5 or SEQ ID NO:6, such peptides may have a sequence which has at least about 30%, or 40%, or 50%, or 60%, or 70%, or 75%, or 80%, or 85%, or 90% or 95% homology to the sequence of a polypeptide having the amino acid sequence of SEQ ID NO:2, SEQ ID NO:3, SEQ ID NO:4, SEQ ID NO:5 or SEQ ID NO:6. Thus, a peptide fragment of any one of the peptides of the invention may include 1, 2, 3, 4, 5, or greater than 5 amino acid alterations.

Moreover, or in addition, the peptide may consist of a truncated version of a polypeptide having the amino acid sequence of SEQ ID NO:2, SEQ ID NO:3, SEQ ID NO:4, SEQ ID NO:5 or SEQ ID NO:6 which has been truncated by 1, 2, 3, 4 or 5 amino acids. A given amino acid may be replaced, for example, by a residue having similar physiochemical characteristics. Examples of such conservative substitutions include substitution of one aliphatic residue for another, such as Ile, Val, Leu, or Ala for one another; substitutions of one polar residue for another, such as between Lys and Arg, Glu and Asp, or Gln and Asn; or substitutions of one aromatic residue for another, such as Phe, Trp, or Tyr for one another. Other conservative substitutions, e.g., involving substitutions of entire regions having similar hydrophobicity characteristics, are well known.

Similarly, polynucleotides of the invention include variants that differ from a native polynucleotide sequence because of one or more deletions, insertions or substitutions, but that encode a biologically active polypeptide. Expression, isolation and purification of polypeptides defining the epitope of the invention and fragments thereof may be accomplished by any suitable technique.

A method for producing polypeptides comprises culturing host cells transformed with a recombinant expression vector encoding a polypeptide having the amino acid sequence of SEQ ID NO:2, SEQ ID NO:3, SEQ ID NO:4, SEQ ID NO:5 or SEQ ID NO:6 under conditions that promote expression of the polypeptide in a disulphide bonded form, then recovering the expressed polypeptides from the culture. The skilled person will recognise that the procedure for purifying the expressed polypeptides will vary according to such factors as the type of host cells employed, and whether the polypeptide is intracellular, membrane-bound or a soluble form that is secreted from the host cell.

Any suitable expression system may be employed. The vectors include a DNA encoding a polypeptide or fragment of the invention, operably linked to suitable transcriptional or translational regulatory nucleotide sequences, such as those derived from a mammalian, avian, microbial, viral, bacterial, or insect gene. Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence. An origin of replication that confers the ability to replicate in the desired (E. coli) host cells, and a selection gene by which transformants are identified, are generally incorporated into the expression vector.

In addition, a sequence encoding an appropriate signal peptide (native or heterologous) can be incorporated into expression vectors. A DNA sequence for a signal peptide (secretory leader) may be fused in frame to the nucleic acid sequence of the invention so that the DNA is initially transcribed, and the mRNA translated, into a fusion protein comprising the signal peptide. A signal peptide that is functional in the intended host cells promotes extracellular secretion of the polypeptide. The signal peptide is cleaved from the polypeptide during translation, but allows secretion of polypeptide from the cell.

Suitable host cells for expression of polypeptides include higher eukaryotic cells and yeast. Prokaryotic systems are also suitable. Mammalian cells, and in particular CHO cells are particularly preferred for use as host cells.

### Administration

A monoclonal antibody or fusion protein of the present invention may be administered alone, but will preferably be administered as a pharmaceutical composition, which will generally comprise a suitable pharmaceutically acceptable excipient, diluent or carrier selected depending on the intended route of administration. Examples of suitable pharmaceutical carriers include; water, glycerol, ethanol and the like.

The antibody of the present invention may be administered to a canine or feline in need of treatment via any suitable route. As detailed herein, it is preferred that the composition is administered parenterally by injection or infusion. Examples of preferred routes for parenteral administration include, but are not limited to, intravenous, intracardial, intraarterial, intraperitoneal, intramuscular, intracavity, subcutaneous, transmucosal, inhalation or transdermal. Routes of administration may further include topical and enteral, for example, mucosal (including pulmonary), oral, nasal and rectal.

In preferred embodiments the composition is deliverable as an injectable composition. For intravenous, intradermal or subcutaneous application, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection or Lactated Ringer's injection. Preservatives, stabilisers, buffers, antioxidants and/or other additives may be included as required. The composition may also be administered via microspheres, liposomes, other microparticulate delivery systems or sustained release formulations placed in certain tissues including blood.

The composition is preferably administered to an individual in a “therapeutically effective amount”, this being sufficient to show benefit to the subject to whom the composition is administered. The actual dose administered, and rate and time-course of administration, will depend on, and can be determined with due reference to, the nature and severity of the condition which is being treated, as well as factors such as the age, sex and weight of the canine to be treated and the route of administration. Further due consideration should be given to the properties of the composition, for example, its binding activity and in-vivo plasma life, the concentration of the fusion protein in the formulation, as well as the route, site and rate of delivery.

Dosage regimens can include a single administration of the composition of the invention, or multiple administrative doses of the composition. The compositions can further be administered sequentially or separately with other therapeutics and medicaments which are used for the treatment of the condition for which the fusion protein of the present invention is being administered to treat.

Examples of dosage regimens which can be administered to a subject can be selected from the group comprising, but not limited to; 1 μg/kg/day through to 20 mg/kg/day, 1 μg/kg/day through to 10 mg/kg/day, 10 m/kg/day through to 1 mg/kg/day.

The actual amount administered, and rate and time-course of administration, will depend on the nature and severity of what is being treated. Prescription of treatment, e.g. decisions on dosage etc, is ultimately within the responsibility and at the discretion of veterinary or medical doctors, and typically takes account of the disorder to be treated, the condition of the canine, the site of delivery, the method of administration and other factors known to practitioners.

### DEFINITIONS

Unless otherwise defined, all technical and scientific terms used herein have the meaning commonly understood by a person who is skilled in the art in the field of the present invention.

Throughout the specification, unless the context demands otherwise, the terms “comprise” or “include”, or variations such as “comprises” or “comprising”, “includes” or “including” will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well as two or more different active agents in combination, while references to “a carrier” includes mixtures of two or more carriers as well as a single carrier, and the like.

The nomenclature used to describe the cyclic polypeptide fragment of the present invention follows the conventional practice wherein the amino group (N) is presented to the left and the carboxy group to the right of each amino acid residue.

The expression “amino acid” as used herein is intended to include both natural and synthetic amino acids, and both D and L amino acids. A synthetic amino acid also encompasses chemically modified amino acids, including, but not limited to salts, and amino acid derivatives such as amides. Amino acids present within the polypeptides of the present invention can be modified by methylation, amidation, acetylation or substitution with other chemical groups which can change the circulating half-life without adversely affecting their biological activity.

The terms “peptide”, “polypeptide” and “protein” are used herein interchangeably to describe a series of at least two amino acids covalently linked by peptide bonds or modified peptide bonds such as isosteres. No limitation is placed on the maximum number of amino acids which may comprise a peptide or protein. Furthermore, the term polypeptide extends to fragments, analogues and derivatives of a peptide, wherein said fragment, analogue or derivative retains the same biological functional activity as the peptide from which the fragment, derivative or analogue is derived.

Furthermore the term “fusion protein” as used herein can also be taken to mean a fusion polypeptide, fusion peptide or the like, or may also be referred to as an immunoconjugate. The term “fusion protein” refers to a molecule in which two or more subunit molecules, typically polypeptides, are covalently or non-covalently linked.

As used herein, the term “therapeutically effective amount” means the amount of a binding agent of the invention which is required to reduce the severity of and/or ameliorate a B-cell mediated disease or at least one symptom thereof.

As used herein, the term “treatment” and associated terms such as “treat” and “treating” means the reduction of the progression, severity and/or duration of a B-cell mediated condition of at least one symptom thereof, wherein said reduction or amelioration results from the administration of a binding compound which has specificity for the CD20 binding epitope of the present invention. The term “treatment” therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition.

As defined herein, a “canine” may also be referred to as a dog. Canines can be categorised as belonging to the subspecies with the trinomial name Canis lupus familiaris (Canis familiaris domesticus) or Canis lupus dingo. Canines include any species of dog and includes both feral and pet varieties, the latter also being referred to as companion animals.

As defined herein, a “feline” may also be referred to as a cat. Felines can be categorised as belonging to the subspecies with the trinomial name Felis silvestris catus. Felines include any species of cat and includes both feral and pet varieties, the latter also being referred to as companion animals.

The present invention will now be described with reference to the following examples which are provided for the purpose of illustration and are not intended to be construed as being limiting on the present invention. The methods and techniques of the present invention are generally performed according to conventional methods well known in the art and as described in various general and more specific references that are cited and discussed throughout the present specification unless otherwise indicated.

### EXAMPLES

### Example 1

### Binding of Murine Anti-Human CD20 Antibodies to the Cyclic Polypeptide of SEQ ID NO:2

The peptide epitope was produced by chemical synthesis and oxidation with an N-terminal biotin moiety attached. This peptide and homologous biotinylated and oxidised human CD20 peptide (IYNCEPANPSEKNSPSTQYC (SEQ ID NO:3) and murine CD20 peptide (IYDCEPSNSSEKNSPSTQYC (SEQ ID NO:6), were coated onto streptavidin coated ELISA plates, then washed and tested for binding of murine anti-human CD20 antibodies.

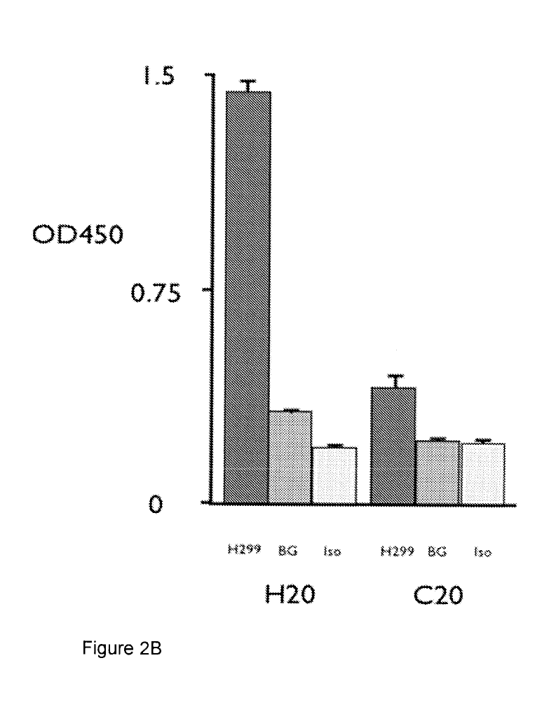

One antibody, B1-H299 (Beckmann Coulter, hereafter referred to as H299 to distinguish it from the commercially available Biogenex B1 monoclonal antibody) was shown to bind specifically to the canine (C20) and human (H20) CD20 peptides, but not to the murine (M20) CD20 peptide. The results of the binding of H299 to the epitope are shown in FIG. 2A. The binding to the canine CD20 peptide epitope was approximately one sixth that of the binding to human CD20 peptide.

Although H299 was shown to bind to canine CD20, Biogenex B1 did not. B1-H299 (H299) and Biogenex B1 (BG) monoclonal antibodies were incubated with canine CD20 peptide or to the homologous human CD20 peptide (IYNCEPANPSEKNSPSTQYC (SEQ ID NO:6)) and binding was detected using a secondary anti-mouse polyclonal HRP conjugate in an ELISA assay. The results are shown in FIG. 2B. Binding to an isotype control antibody is shown (Iso). It is not known whether the reduced binding of the Biogenex B1 monoclonal antibody to peptide when compared with H299 is due to lack of affinity or whether it simply reflects inaccuracy in the concentration of the Biogenex monoclonal antibody preparation supplied.

### Example 2

### H299 Monoclonal Antibody and Intact Cellular CD20 Binding Study

In order to confirm that the binding of the H299 monoclonal antibody to the canine CD20 epitope loop was sufficient for binding to intact cellular CD20, the canine and human CD20 were expressed on the surface of CHO cells and tested for binding of H299 by fluorescence activated cell sorter (FACS).

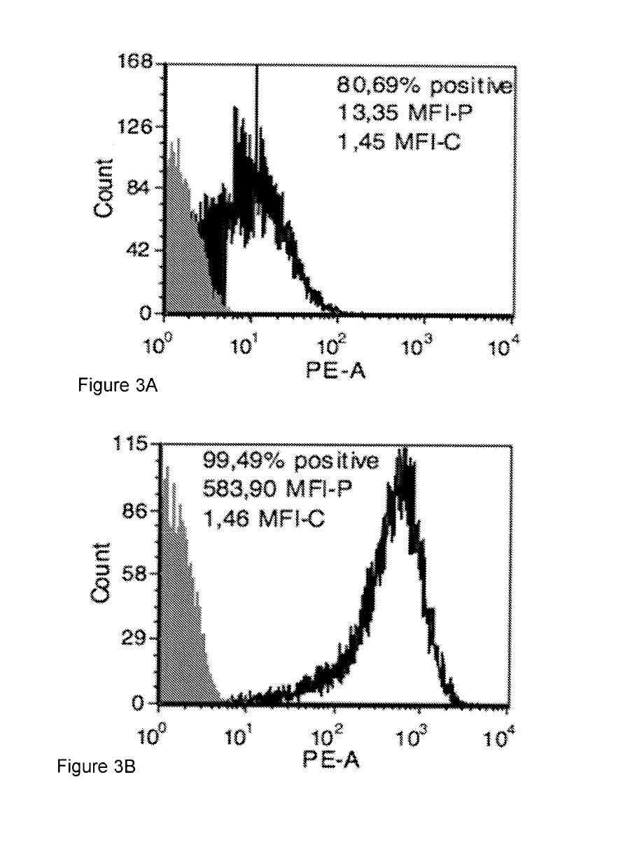

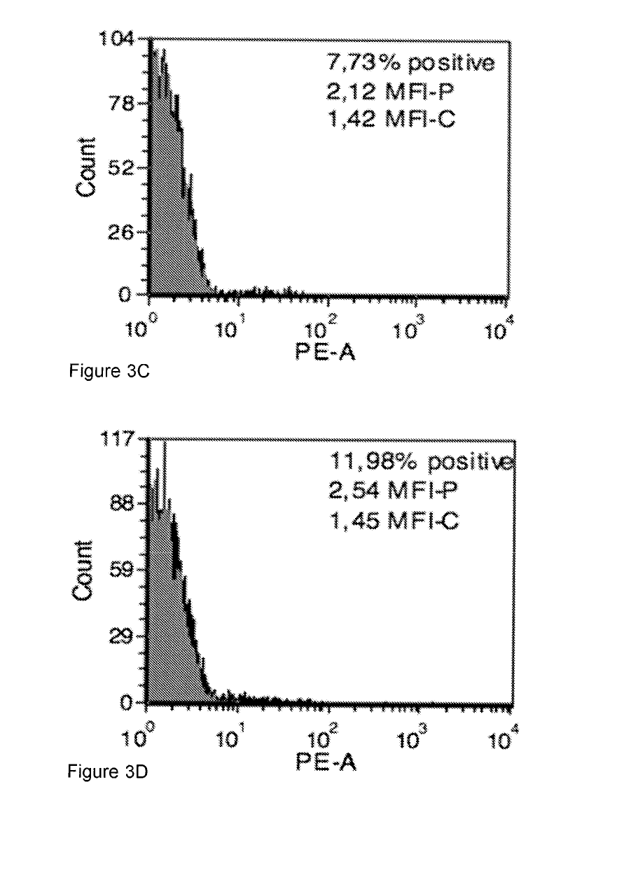

H299 binds both intact human and canine CD20 expressed on CHO cells. The results are shown in FIGS. 3A-D. FIG. 3A shows the binding of H299 (6 μg/ml) to canine CD20 CHO cells. FIG. 3B shows the binding of H299 (6 μg/ml) to human CD20 CHO cells. FIG. 3C shows the binding of secondary anti-mouse PE antibody (Sigma P9287) alone to canine CD20 CHO cells. FIG. 3D shows the binding of secondary anti-mouse PE antibody (Sigma P9287) alone to human CD20 CHO cells.

### Example 3

### Comparison of the Binding of Bly1 and H299 Anti-Human CD20 Monoclonal Antibodies to Canine CD20 Peptide SEQ ID NO:2

Binding of Bly1 and H299 monoclonal antibodies to the oxidised peptide loop of canine CD20 (SEQ ID NO:2) was compared by ELISA and on the surface of transfected CHO cells by FACS. Microtitre plate wells were coated with oxidised peptide at 50 ug/ml. H299 or Bly1 MAbs were added at increasing concentrations from 1.25-10 ug/ml and developed using polyclonal anti-mouse IgG horseradish peroxidase (HRP).

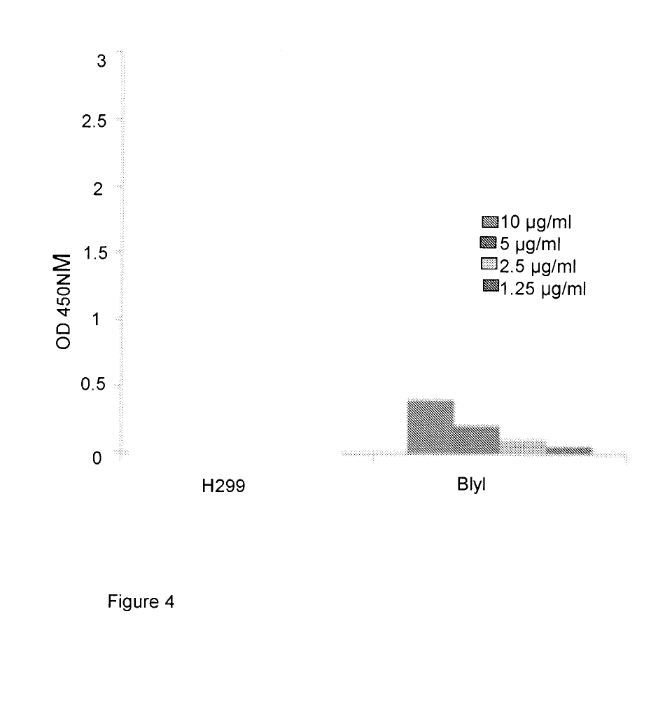

The results are shown in FIG. 4. The H299 monoclonal antibody bound with higher affinity than the Bly1 monoclonal antibody to the oxidised canine CD20 peptide under these conditions.

### Example 4

### H299 and Bly1 monoclonal Antibodies and Intact Cellular CD20 Binding Study

In order to confirm that the binding of the Type II anti-human CD20 monoclonal antibodies H299 and Bly1 to the canine CD20 epitope loop was sufficient for binding to intact cellular CD20, the canine and human CD20 polypeptides (SEQ ID NO:2 and SEQ ID NO:3) were expressed on the surface of CHO cells and tested for binding of H299 or Bly1 as indicated by fluorescence intensity and the percentage of positive cells in the sample using a fluorescence activated cell sorter (FACS).

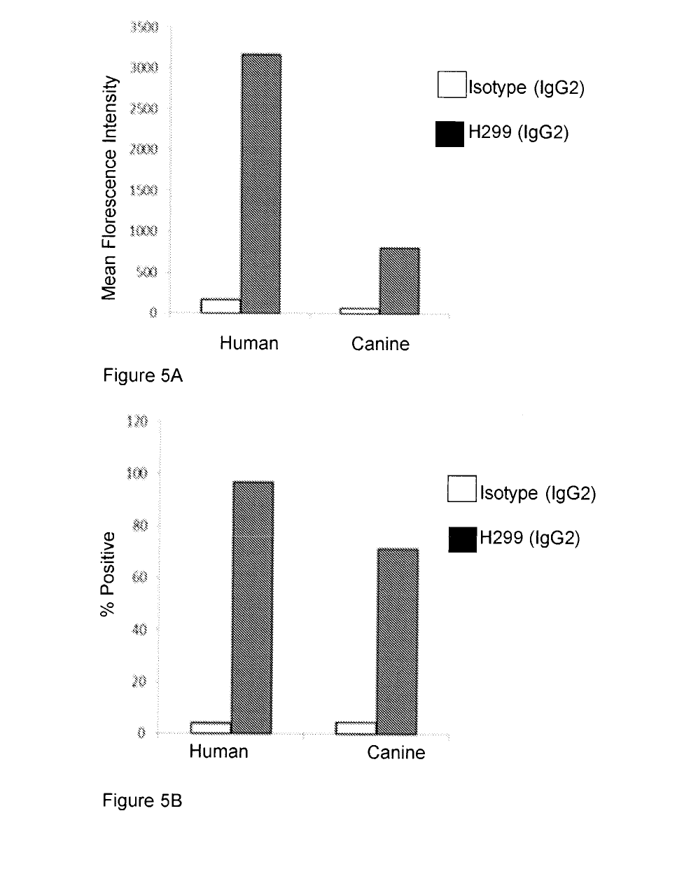

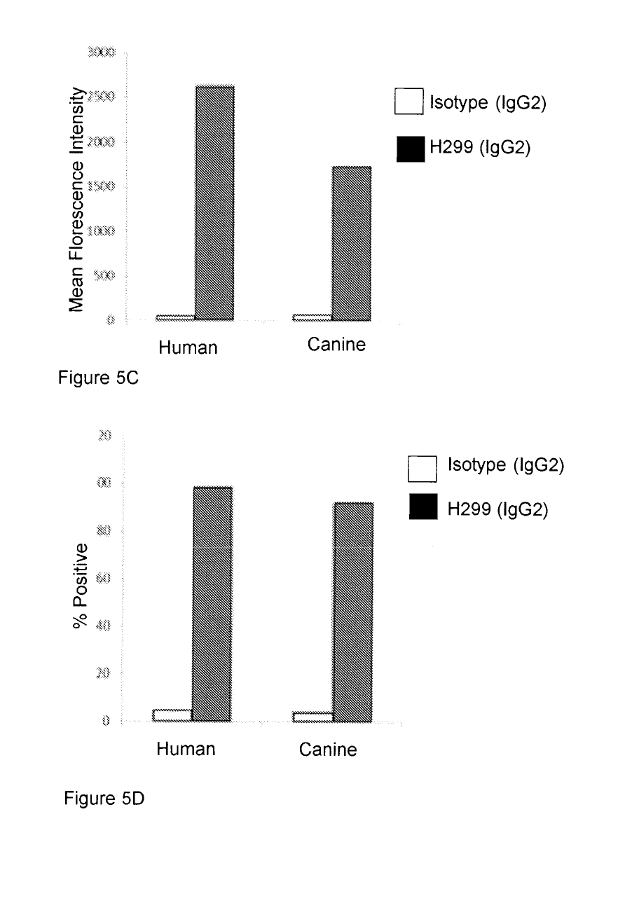

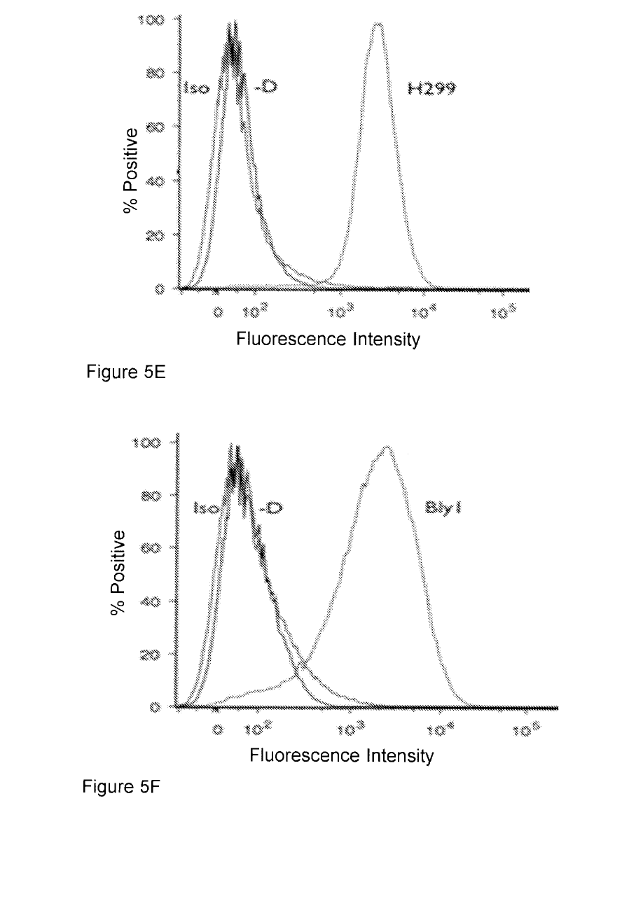

The results are shown in FIGS. 5A-F. The Type II anti-human CD20 monoclonal antibodies H299 and Bly 1 bind human and canine CD20 expressed on CHO cells to a similar extent. FIGS. 5A and 5B show the binding of the H299 monoclonal antibody (10 ug/ml) to canine and human CD20 CHO cells as indicated by mean fluorescence intensity (FIG. 5A) and by the percentage of positive cells (FIG. 5B). FIGS. 5C and 5D show the binding of the Bly1 monoclonal antibody (10 ug/ml) to canine and human CD20 CHO cells as indicated by mean fluorescence intensity (FIG. 5C) and by the percentage of positive cells (FIG. 5D). FIGS. 5E and 5F show FACS histograms of binding of H299 monoclonal antibody and Bly1 monoclonal antibody respectively to canine CD20-CHO cells; isotype control (Iso) and samples prepared without detection antibody (-D) are indicated.

### Example 5

### Demonstration that the Binding to Canine CD20 by Type II Monoclonal Antibody H299 is Critically Dependent on Disulphide Bonding

The biotinylated oxidised canine peptide SEQ ID NO:2 was bound to the surface of streptavidin-coated microtitre plate wells alongside wells coated with the homologous biotinylated human CD20 peptide (IYNCEPANPSEKNSPSTQYC (SEQ ID NO:3)). Half of the peptide coated wells were treated with dithiothreitol (DTT) to reduce the disulphide bonds and then washed. The binding of the H299 monoclonal antibody was then assessed either in the presence or absence of continuing DTT.

An ELISA plate (Reacti-Bind NeutrAvidin Coated with blocker BSA, Thermo Scientific, catalogue number 15123) was incubated overnight at 4° C. with 100 μl or 50 μg/ml of human or canine CD20 peptides in PBS. The plate was washed three times with PBS and the peptides reduced by incubating with 100 μl of 15 mM DTT in PBS, at room temperature, for 30 minutes. The PBS/DTT solution was removed and the plate was incubated with 100 μl of B1 monoclonal antibody at 500 ng/ml in either PBS or 5 mM DTT/PBS, for 1 hour at room temperate. Mouse IgG2a K Isotype Control (ebiosciences, catalogue number 16-4724-81) was used as control antibody at 500 ng/ml. The plate was washed three times with PBS and incubated with 100 μl detection antibody (Sigma, Polyclonal Rabbit Anti-Mouse immunoglobulins/HRP catalogue number P 0161) diluted to 1 in 5000 in PBS. After washing three times with PBS, 100 μl of substrate was added and the reaction allowed to develop. Stop solution was added (100 μl, 2M H2S04) and the absorbance of the wells was read at 450 nm.

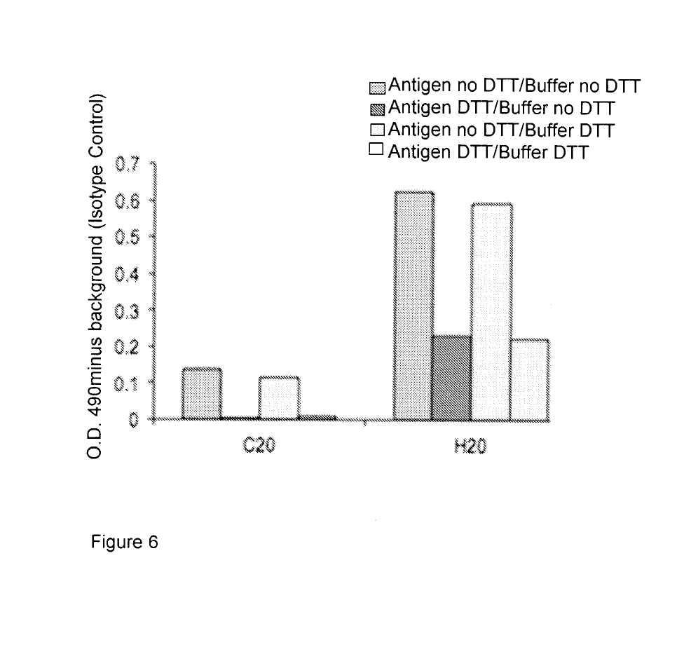

The results are shown in FIG. 6 and these show that the binding of H299 to the canine CD20 epitope is critically dependent on it being oxidised, since pre-treatment with DTT abolished all binding to the canine peptide. Binding to the homologous human CD20 peptide was reduced, but not eliminated by oxidation and this is consistent with the ability of H299 to bind to linear peptides of the same region described previously (Teeling et al., 2006) in which the cysteine residues were each replaced by Alanine. The lack of binding of H299 to linear (reduced) canine peptide loop is consistent with the failure of the prior art to detect binding of anti-human CD20 monoclonal antibodies to a linear, larger peptide of the putative canine CD20 extracellular domain (Beall et al.). It is also consistent with the findings of Niederfellner at al. (2011) which showed binding of H299 to linear peptides (8 mers) derived from the human CD20 sequence.

### Example 6

### Example of Caninised Form of the GA101 Monoclonal Antibody

In this example, the inventor caninised the Type II anti-human CD20 monoclonal antibody GA101 by way of substituting framework region amino acid sequences in order to reduce the immunogenicity of the antibody when it is administered to canines. No alteration was made to the amino acid sequence of the CDR domains. Furthermore, the “caninised” heavy and light chain variable domains were conjoined to canine derived constant domains.

The amino acid sequence of the caninised GA101 light chain variable domain is shown in SEQ ID NO:15, with SEQ ID NO:7-10 (FR1-DIVMTQTPLSLSVTPEEPASISC (SEQ ID NO:7), FR2-WYLQKPGQSPQLLIY (SEQ ID NO:8), FR3-GVPDRFSGSGSGTDFTLKISRVEAEDVGVYYC (SEQ ID NO:9), FR4-FGAGTKVEIK (SEQ ID NO:10), showing the framework regions (FR1 to FR4).

Further, the amino acid sequence of the caninised GA101 variable domain is shown in SEQ ID NO:16, with SEQ ID NO:11-14 (FR1-EVQLVQSGGEVVKPGGSLKVSCVASGF (SEQ ID NO:11), FR2-WVRQAPGQGMEWVG (SEQ ID NO:12), FR3-RVTITRDNSKSTAYLELSSLRSEDTAVYYCAR (SEQ ID NO:13), FR4-WGQGTLVTVSS (SEQ ID NO:14)) showing the framework regions (FR1 to FR4).

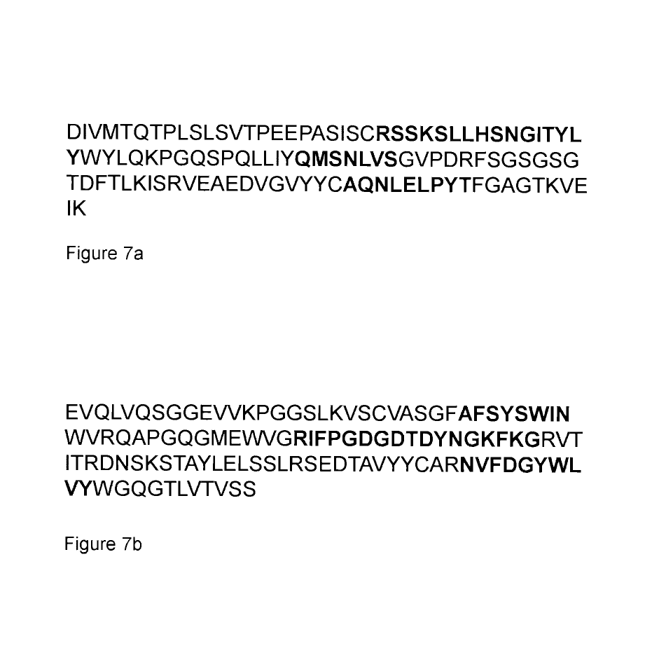

FIG. 7 shows the complete caninised GA101 VK kappa variable light chain amino sequence (SEQ ID NO:15) wherein the CDR region residues are shown in bold and also the complete caninised GA101 VH variable heavy chain (SEQ ID NO:16) wherein the CDR region residues are shown in bold. Furthermore, Tables 1-8 show the amino acid residue substitutions used in the process of caninising the GA101 monoclonal antibody light and heavy chain variable domain framework regions.

TABLE 1
Light chain variable domain FR1 residues

| Light | Kabat light | | | |
|---|---|---|---|---|
| chain | chain | | | Canin- |
| FR1 | numbering | Canine | | ised |
| position | position | VK | GA101 | GA101 |
| 1 | 1 | D | D | D |
| 2 | 2 | I | I | I |
| 3 | 3 | V | V | V |
| 4 | 4 | M | M | M |
| 5 | 5 | MTI | T | T |
| 6 | 6 | Q | Q | Q |
| 7 | 7 | TS | T | T |
| 8 | 8 | P | P | P |
| 9 | 9 | LAP | L | L |
| 10 | 10 | S | S | S |
| 11 | 11 | L | L | L |
| 12 | 12 | SA | P | S |
| 13 | 13 | VL | V | V |
| 14 | 14 | STR | T | T |
| 15 | 15 | PQR | P | P |
| 16 | 16 | ED | G | E |
| 17 | 17 | E | E | E |
| 18 | 18 | PTLKEA | P | P |
| 19 | 19 | AV | A | A |
| 20 | 20 | ST | S | S |
| 21 | 21 | I | I | I |
| 22 | 22 | STY | S | S |
| 23 | 23 | CY | C | C |

TABLE 2
Light chain variable domain FR2 residues

| Light | Kabal light | | | |
|---|---|---|---|---|
| Chain | chain | | | Canin- |
| FR2 | numbering | Canine | | ised |
| Position | system | VK | GA101 | GA101 |
| 1 | 35 | W | W | W |
| 2 | 36 | FYIL | Y | Y |
| 3 | 37 | RQLI | L | L |
| 4 | 38 | QH | Q | Q |
| 5 | 39 | KR | K | K |
| 6 | 40 | PSA | P | P |
| 7 | 41 | GD | G | G |
| 8 | 42 | Q | Q | Q |
| 9 | 43 | SATP | S | S |
| 10 | 44 | P | P | P |
| 11 | 45 | QKER | Q | Q |
| 12 | 46 | RLPGAS | L | L |
| 13 | 47 | L | L | L |
| 14 | 48 | IL | I | I |
| 15 | 49 | YFNSEV | Y | Y |

TABLE 3
Light chain variable domain FR3 residues

| Light | Kabat light | | | |
|---|---|---|---|---|
| chain | chain | | | Canin- |
| FR3 | numbering | Canine | | ised |
| position | position | VK | GA101 | GA101 |
| 1 | 57 | GA | G | G |
| 2 | 58 | VA | V | V |
| 3 | 59 | PS | P | P |
| 4 | 60 | DS | D | D |
| 5 | 61 | R | R | R |
| 6 | 62 | FLV | F | F |
| 7 | 63 | SI | S | S |
| 8 | 64 | GA | G | G |
| 9 | 65 | S | S | S |
| 10 | 66 | G | G | G |
| 11 | 67 | S | S | S |
| 12 | 68 | G | G | G |
| 13 | 69 | TA | T | T |
| 14 | 70 | DE | D | D |
| 15 | 71 | F | F | F |
| 16 | 72 | TSR | T | T |
| 17 | 73 | LF | L | L |
| 18 | 74 | RTK | K | K |
| 19 | 75 | I | I | I |
| 20 | 76 | S | S | S |
| 21 | 77 | RGST | R | R |
| 22 | 78 | VL | V | V |
| 23 | 79 | E | E | E |
| 24 | 80 | AP | A | A |
| 25 | 81 | DEGIN | E | E |
| 26 | 82 | D | D | D |
| 27 | 82A | AVTGS | V | V |
| 28 | 82B | GA | G | G |
| 29 | 82C | VIL | V | V |
| 30 | 83 | Y | Y | Y |
| 31 | 84 | Y | Y | Y |
| 32 | 85 | C | C | C |

TABLE 4
Light chain variable domain FR4 residues

| Light | Kabat light | | | |
|---|---|---|---|---|
| chain | chain | | | Canin- |
| FR4 | numbering | Canine | | ised |
| position | position | VK | GA101 | GA101 |
| 1 | 95 | F | F | F |
| 2 | 96 | GS | G | G |
| 3 | 97 | AQPT | G | A |
| 4 | 98 | GE | G | G |
| 5 | 99 | TP | T | T |
| 6 | 100 | KQS | K | K |
| 7 | 101 | VLW | V | V |
| 8 | 102 | DER | E | E |
| 9 | 103 | LI | I | I |
| 10 | 104 | K | K | K |

TABLE 5
Heavy chain variable domain FR1 residues

| Heavy | Kabat heavy | | | |
|---|---|---|---|---|
| chain | chain | | | Canin- |
| FR1 | numbering | Canine | | ised |
| position | position | VH | GA101 | GA101 |
| 1 | 1 | EDG | Q | E |
| 2 | 2 | VGLEIM | V | V |
| 3 | 3 | QHRAV | Q | Q |
| | | EKLPS | | |
| 4 | 4 | LVP | L | L |
| 5 | 5 | VALEM | V | V |
| 6 | 6 | EQA | Q | Q |
| 7 | 7 | SFLT | S | S |
| 8 | 8 | G | G | G |
| 9 | 9 | GE | A | G |
| 10 | 10 | DAGNET | E | E |
| 11 | 11 | LQRVW | V | V |
| 12 | 12 | VAIM | K | V |
| 13 | 13 | KRNQ | K | K |
| 14 | 14 | PFT | P | P |
| 15 | 15 | GAET | G | G |
| 16 | 16 | GEA | S | G |
| 17 | 17 | STP | S | S |
| 18 | 18 | LR | V | L |
| 19 | 19 | RKTGV | K | K |
| 20 | 20 | LIV | V | V |
| 21 | 21 | SY | S | S |
| 22 | 22 | C | C | C |
| 23 | 23 | VLAIE | K | V |
| 24 | 24 | ATVGIS | A | A |
| 25 | 25 | SPGT | S | S |
| 26 | 26 | GDRT | G | G |
| 27 | 27 | FLIDST | Y | F |
| | | V | | |

TABLE 6
Heavy chain variable domain FR2 residues

| Heavy | Kabat heavy | | | |
|---|---|---|---|---|
| chain | chain | | | Canin- |
| FR2 | numbering | Canine | | ised |
| position | system | VH | GA101 | GA101 |
| 1 | 36 | WC | W | W |
| 2 | 37 | VIAFL | V | V |
| 3 | 38 | R | R | R |
| 4 | 39 | QLH | Q | Q |
| 5 | 40 | ASTGPVD | A | A |
| 6 | 41 | PL | P | P |
| 7 | 42 | GERL | G | G |
| 8 | 43 | KREGAMQ | Q | Q |
| 9 | 44 | GERDTV | G | G |
| 10 | 45 | TPFM | L | M |
| 11 | 46 | QEHDLPR | E | E |
| 12 | 47 | WLCSYFM | W | W |
| 13 | 48 | VLIF | M | V |
| 14 | 49 | ATSGL | G | G |

TABLE 7
Heavy chain variable domain FR3 residues

| Heavy | Kabat heavy | | | |
|---|---|---|---|---|
| chain | chain | | | Canin- |
| FR3 | numbering | Canine | | ised |
| position | position | VH | GA101 | GA101 |
| 1 | 66 | RQ | R | R |
| 2 | 67 | FVL | V | V |
| 3 | 68 | TAIS | T | T |
| 4 | 69 | IVLMT | I | I |
| 5 | 70 | SAFT | T | T |
| 6 | 71 | RK | A | R |
| 7 | 72 | DEN | D | D |
| 8 | 73 | NDTSIG | K | N |
| 9 | 74 | AGVSDP | S | S |
| | | T | | |
| 10 | 75 | KRENQ | T | K |
| | | GM | | |
| 11 | 76 | NDSKHR | S | S |
| 12 | 77 | TMIAS | T | T |
| 13 | 78 | LVMAI | A | A |
| 14 | 79 | YFSHT | Y | Y |
| 15 | 80 | LI | M | L |
| 16 | 81 | QHEDRA | E | E |
| 17 | 82 | ML | L | L |
| 18 | 82A | NDSTH | S | S |
| | | KPR | | |
| 19 | 82B | SGDRNT | S | S |
| 20 | 82C | LV | L | L |
| 21 | 83 | RTGKSI | R | R |
| 22 | 84 | AVDTSG | S | S |
| | | P | | |
| 23 | 85 | EDAV | E | E |
| 24 | 86 | D | D | D |
| 25 | 87 | TASM | T | T |
| 26 | 88 | AGV | A | A |
| 27 | 89 | VMILFT | V | V |
| | | KQ | | |
| 28 | 90 | YH | Y | Y |
| 29 | 91 | YFH | Y | Y |
| 30 | 92 | C | C | C |
| 31 | 93 | AVTGM | A | A |
| | | RSCL | | |
| 32 | 94 | KRSNG | R | R |
| | | ATPDQ | | |
| | | VEIM | | |

TABLE 8
Heavy chain variable domain FR4 residues

| Heavy | Kabat heavy | | | |
|---|---|---|---|---|
| chain | chain | | | Canin- |
| FR4 | numbering | Canine | | ised |
| position | position | VH | GA101 | GA101 |
| 1 | 103 | WL | W | W |
| 2 | 104 | GAS | G | G |
| 3 | 105 | QPHRD | Q | Q |
| 4 | 106 | G | G | G |
| 5 | 107 | TASIN | T | T |
| 6 | 108 | LSQPR | L | L |
| 7 | 109 | VLIP | V | V |
| 8 | 110 | TFIASL | T | T |
| | | PY | | |
| 9 | 111 | VA | V | V |
| 10 | 112 | SACPT | S | S |
| 11 | 113 | SLAP | S | S |

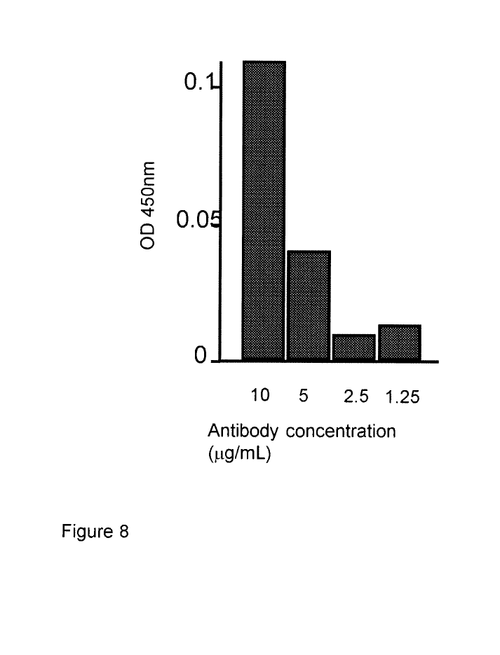

DNA encoding full length caninised GA-101 heavy and light chains (SEQ ID NO:17 and SEQ ID NO:18) was transfected into CHO cells and the supernatant tested for binding by ELISA to canine CD20 cyclic peptide SEQ ID NO:2. The dose response indicates that like other Type II anti-human CD20 antibodies, and its parent mouse antibody Bly-1, the caninised GA101 also binds to canine CD20. FIG. 8 shows that when expressed as a complete canine antibody the caninised variable domains of GA101 bind to the cyclic canine CD20 peptide of FIG. 1 (SEQ ID NO:2). This confirms the data with Bly-1 above (and by extension its humanised variant GA101) that this is a further example of a Type II anti-human CD20 monoclonal antibody that also binds to a common epitope on canine CD20, thus validating the use of the structurally constrained shared epitope on canine CD20 as a novel target for monoclonal antibodies.

### Example 7

### Novel Type II Monoclonal Antibodies RA2 and RF2

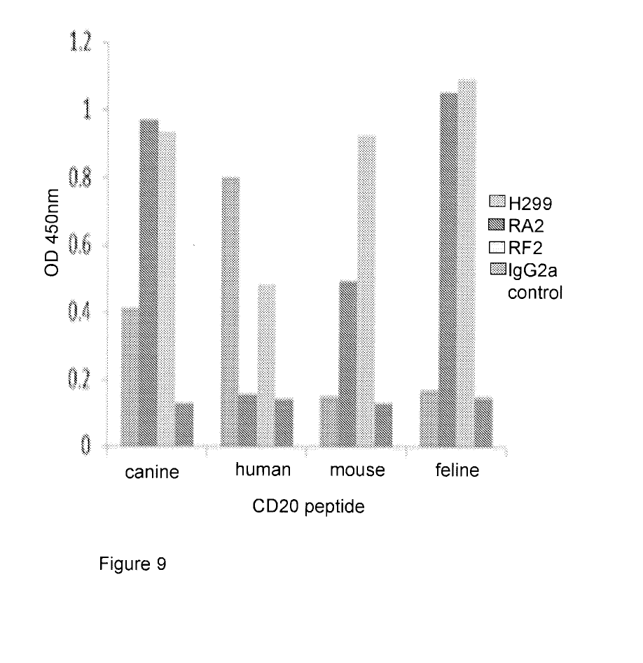

Using a combination of cyclic human (SEQ ID NO:3) and canine (SEQ ID NO:2) CD20 peptides, monoclonal antibodies that would bind both human and canine peptides were screened for using conventional hybridoma screening of mice previously immunised with the cyclic canine CD20 peptide of FIG. 1, coupled to diphtheria toxoid at the amino terminus as a source of T-cell help. Two antibody hybridomas were selected, RA2 and RF2 and tested for binding to these as well as to murine (SEQ ID NO:6) and feline (SEQ ID NO:4) CD20 peptides. The results are shown in FIG. 9. RA2 and RF2 bound strongly to canine CD20 and RF2 bound strongly to human CD20 peptide whereas RA2 binds more weakly. Unexpectedly RA2 and RF2 bound strongly to feline and also murine CD20 peptides. This novel binding pattern suggests an overlapping epitope with that of the Type II anti-human antibody H299 (also shown in FIG. 9).

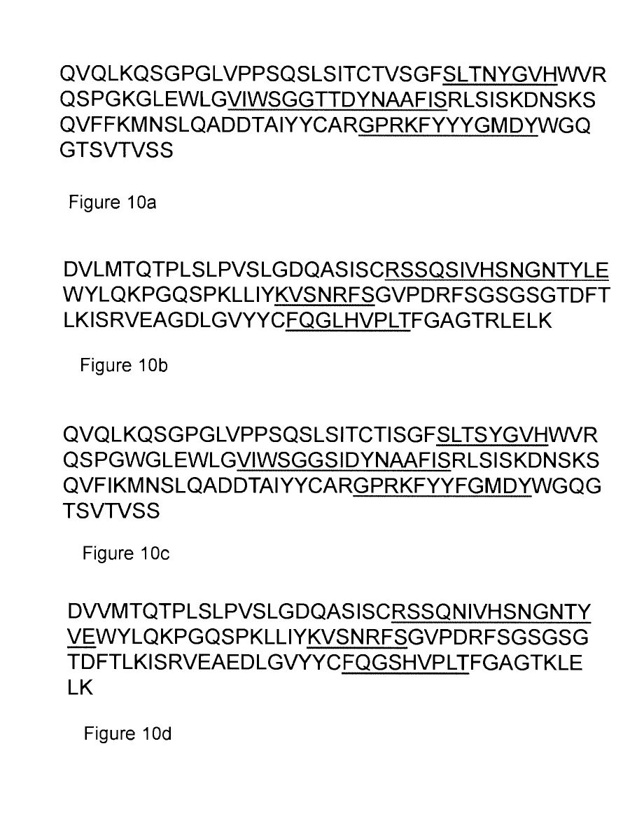

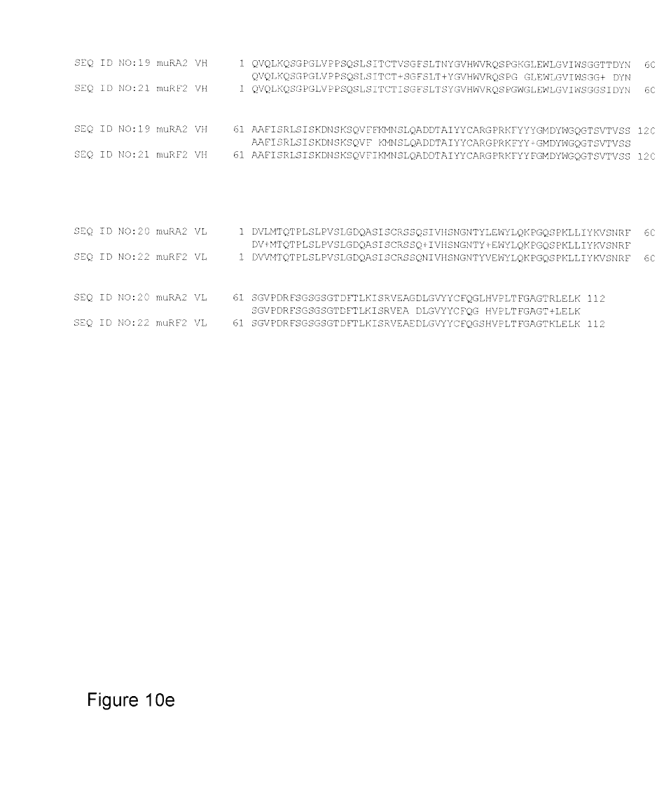

The mRNA encoding the variable heavy and light chains of RA2 and RF2 was extracted using a Qiagen kit and cDNA were prepared by RT-PCR using mouse immunoglobulin specific oligonucleotide primers (Novagen) and a Superscript III first strand synthesis system kit. The PCR products were sequenced using Novagen IgG kappa specific constant domain reverse primers. The derived sequences are shown in FIG. 10 (a,b muRA2 VH and VL; c,d muRF2 VH and VL. The sequences are related (FIG. 10 e), but with several differences in the CDR regions.

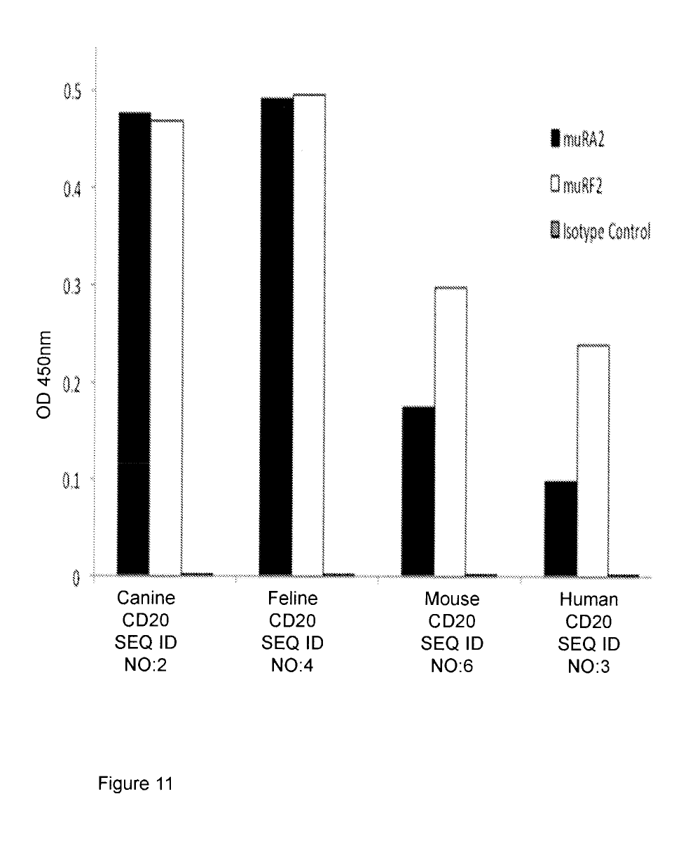

The RA2 and RF2 variable heavy and light chain sequences (SEQ ID NO:23-26) were rebuilt as IgG2a/kappa antibodies by oligonucleotide-based gene synthesis, cloned into pcDNA3.3 vectors and co-expressed in appropriate pairs in CHO cells. The supernatant expressed antibodies were purified by Protein A chromatography and tested for binding by CD20 peptide ELISA (FIG. 11). Both purified recombinant forms of mouse RA2 and RF2 bound strongly to cyclic canine and feline CD20 peptides, and also to mouse and human CD20 peptides (the binding of RA2 to human CD20 peptide was more pronounced than with the corresponding hybridoma supernatant in FIG. 9).

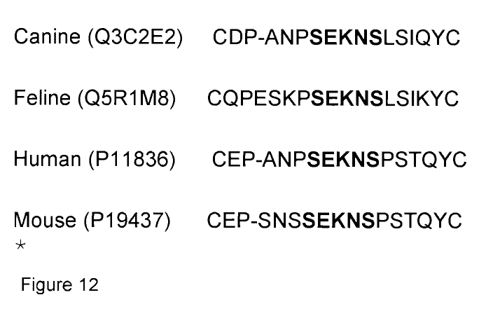

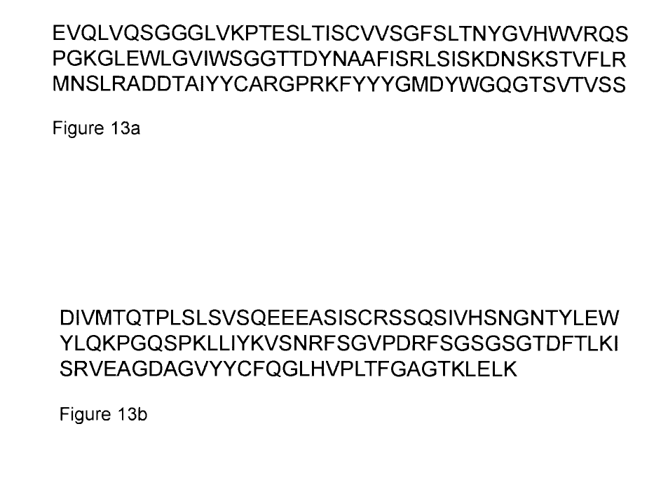

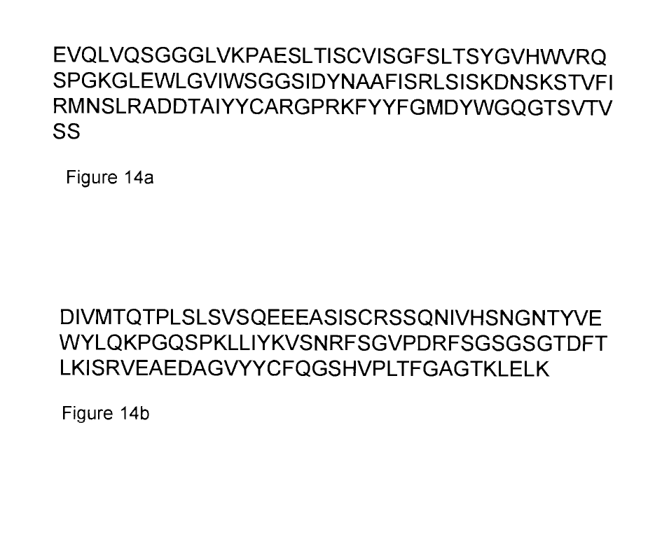

Thus confirmation of binding of RA2 and RF2 antibodies to cyclic canine, human, mouse and feline CD20 epitopes suggests a more constrained epitope than that shared by the Type II anti-human CD20 antibodies exemplified by H299 and Bly1. FIG. 12 illustrates an alignment of the corresponding CD20 peptides from each species, from which the common epitope SEKNS can be derived (SEQ ID NO:67). SEKNS (SEQ ID NO:67) is a sub-epitope of PSEKNSL (SEQ ID NO:1) suggesting an unexpectedly small epitope common to each of these species was derived by immunisation and selection for RA2 and RF2.

In order to make RA2 and RF2 useful in therapy of canines and felines, chimeric canine, caninised and felinised antibodies were designed (as per caninised GA101 above) and expressed and purified from CHO cells using the techniques described above for the expression and purification of recombinant mouse RA2 and RF2.

### Example 8

### Chimeric Canine and Caninised RA2 and RF2 Antibodies

Chimeric mouse-canine forms of RA2 heavy (isotypes A,B,C,D) and light chains are described by SEQ ID NO:27, 28, 29, 30 and 31.

Chimeric mouse-canine forms of RF2 heavy (isotypes A,B,C,D) and light chains are described by SEQ ID NO:32, 33, 34, 35 and 36.

Caninised RA2 variable heavy (VH) and light (VL) domains are described by SEQ ID NO:37 and 38. Full caninised RA2 antibody heavy (isotypes A,B,C,D) and light chains are described by SEQ ID NO:40, 41, 42, 43 and 44.

Caninised RF2 variable heavy (VH) and light (VL) domains are described by SEQ ID NO:44 and 45. Full caninised RF2 antibody heavy (isotypes A,B,C,D) and light chains are described by SEQ ID NO:46, 47, 48, 49 and 50.

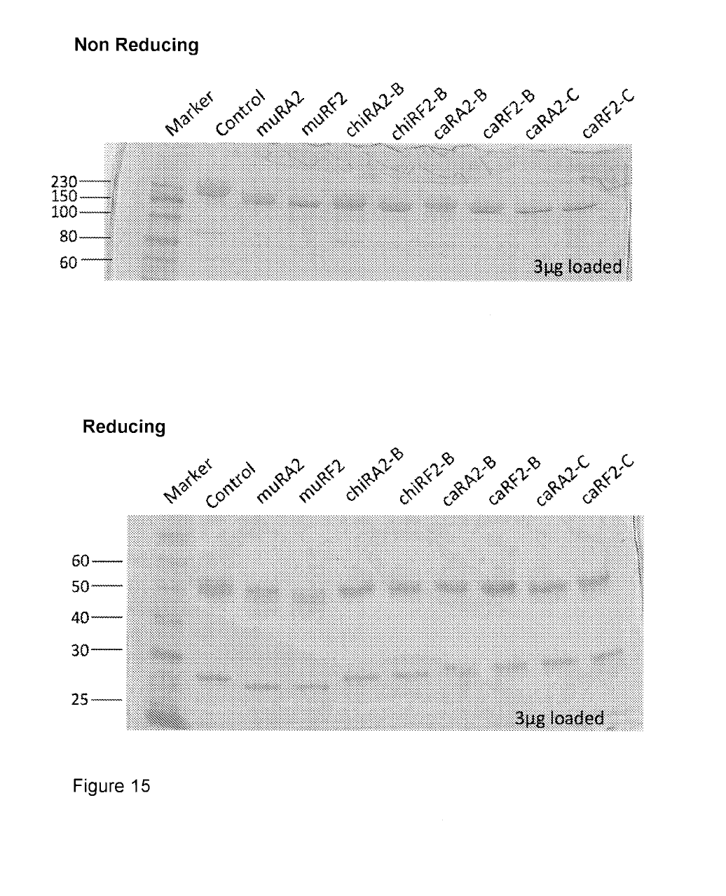

FIG. 15 shows a SDS-PAGE gel experiment in which purified mouse RA2 and RF2, chimeric RA2 (HCB Isotype SEQ ID NO:28 and 31) and RF2 (HCB isotype SEQ ID NO:33 and 36) and caninised RA2 (HCB isotype SEQ ID NO:40 and 43; HCC Isotype SEQ ID NO:41 and 43) and RF2 antibodies (HCB isotype SEQ ID NO:47 and 50; HCC isotype SEQ ID NO:48 and 50) were separated under reducing and non-reducing conditions. HCB and HCC isotypes were selected for their ability to recruit complement, a desirable feature in antibodies with potential use for example in the treatment and elimination of CD20-expressing lymphoma cells in vivo in dogs. Tetrameric H2L2 antibodies are shown in the upper non-reducing gel at approximately 150 kDa molecular weight. Bands corresponding to heavy and light chains can be seen in the lower reducing gel at approximately 50 kDa and 25 kDa respectively.

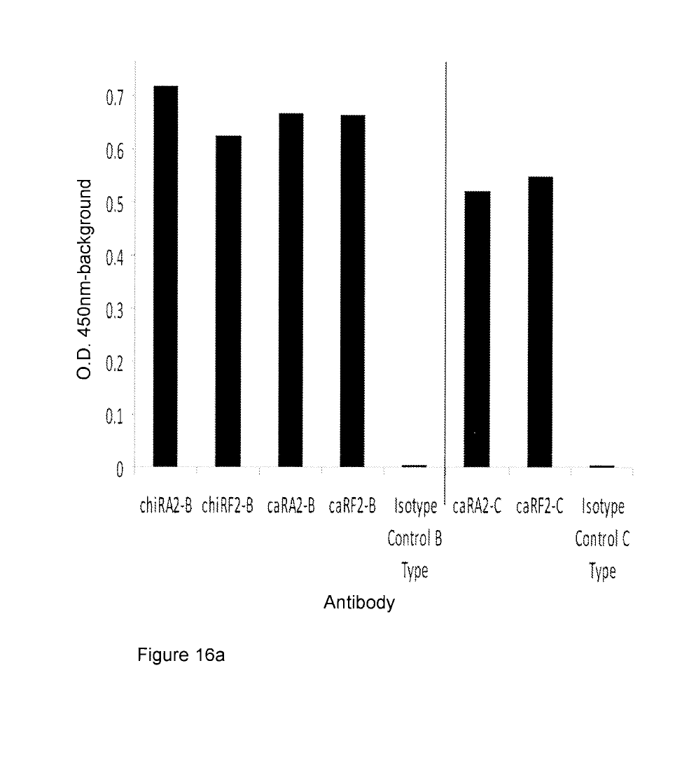

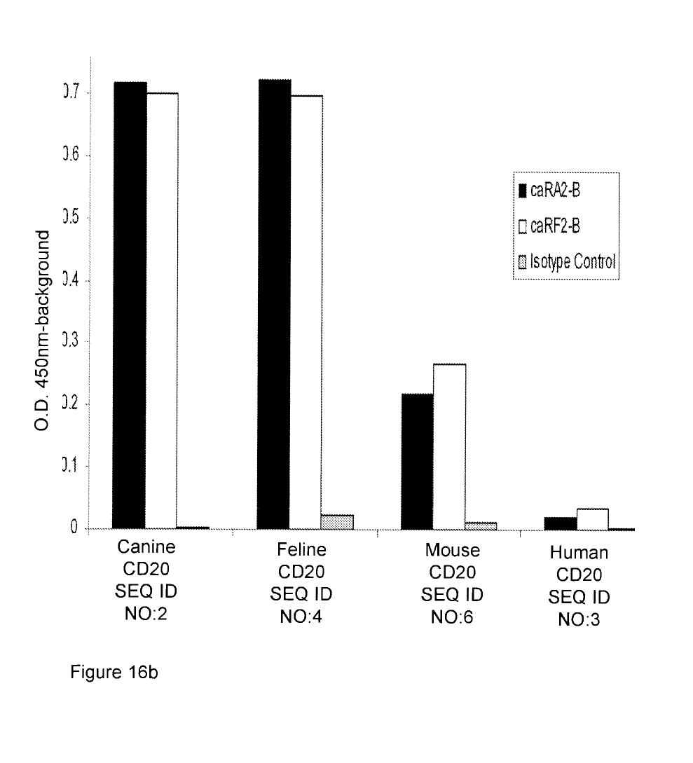

The purified antibodies shown in FIG. 15 were tested for binding to cyclic CD20 peptides from different species by ELISA as above. FIG. 16a shows binding of chimeric and caninised RA2 and RF2 antibodies to cyclic canine CD20 peptide. FIG. 16b shows binding to peptides from different species with binding specificities broadly similar to those of the parent mouse antibodies (c.f FIG. 11).

### Example 9

### Chimeric and Caninised RA2 and RF2 anti-CD20 Antibodies have Active Fc Domains

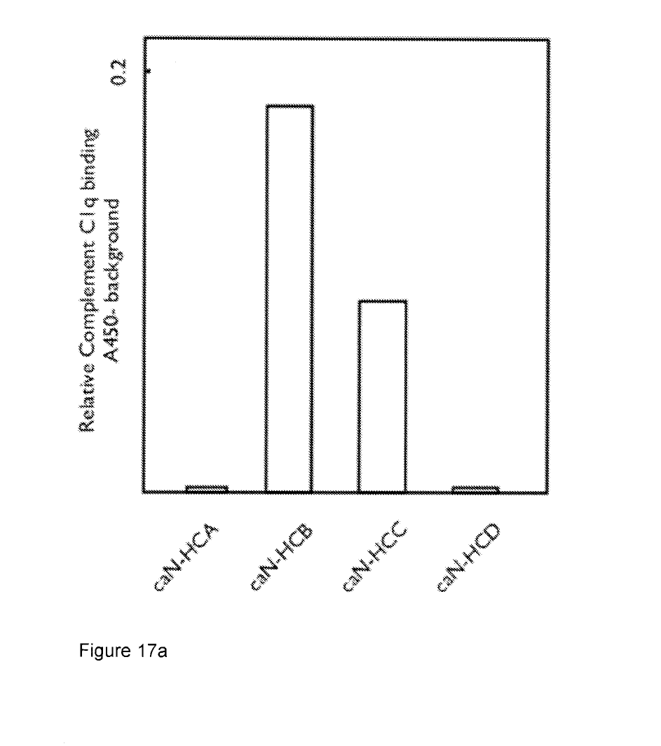

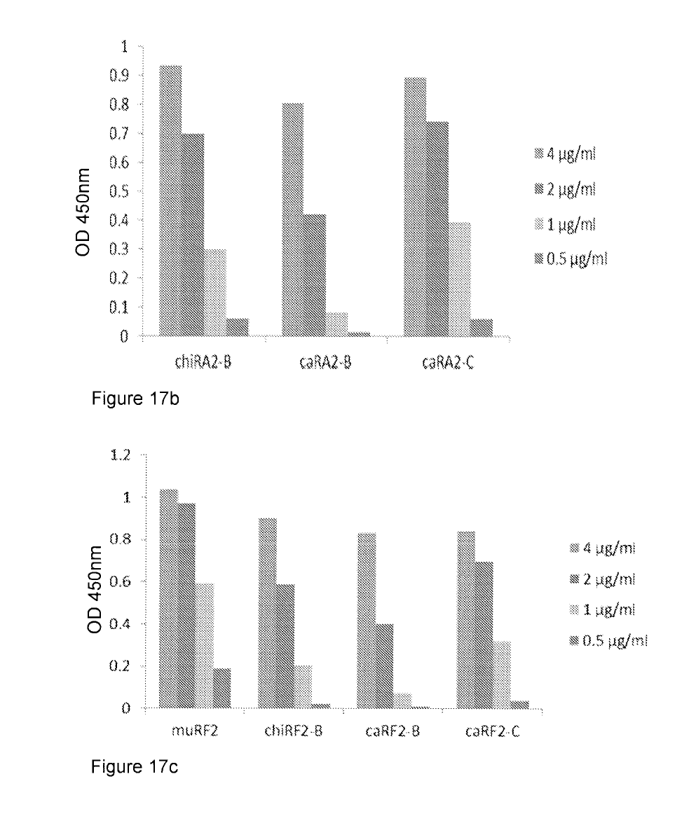

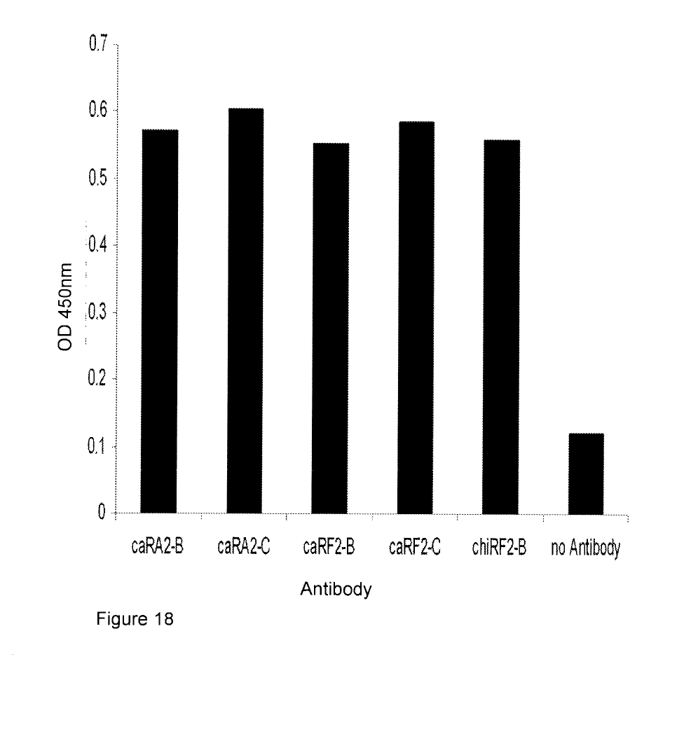

Mechanisms that enable the elimination of canine CD20-expressing cells in therapy of disease (e.g. in dogs suffering from canine CD20+ lymphoma, or from B cell-mediated inflammatory diseases) include recruitment of the effector arms of the immune system following binding of anti-CD20 antibodies. FIGS. 17 and 18 demonstrate that the selected canine heavy chains HCB and HCC used in construction of the recombinant chimeric and caninised RA2 and RF2 antibodies are functional in recruiting complement (FIG. 17, C1q ELISA) and in binding to canine CD64, the high affinity Fc receptor which mediates antibody dependent cellular cytotoxicity (FIG. 18). FIG. 17 shows binding of complement C1q to monoclonal antibodies constructed using four different heavy chain isotypes (HCA, HCB, HCC, HCD). Panel A shows, by way of example, that anti-canine NGF antibodies bearing canine HCA and HCD isotypes do not recruit complement, whereas those with HCB and HCC do recruit complement. Hence, we constructed the recombinant anti-canine CD20 antibodies (for which effector function is desirable) as HCB and HCC isotypes. As can be seen from Panels B and C, both the mouse IgG2a variant of RF2 (muRF2) and each of the chimeric HCB (chiRA2-B, chiRF2-B), caninised HCB (caRA2-B, caRF2-B) and caninised HCC (caRA2-C, caRF2-C) isotype variants bind to complement as evidenced by the positive immunoreactivity to complement C1q by ELISA. Binding to C1q indicates that these antibodies will mediate complement-dependent cytotoxicity (CDC). All forms of chimeric and caninised antibodies based on the HCB and HCC isotype were positive in this assay.

FIG. 18 shows binding of soluble recombinant canine high affinity Fc receptor (scaCD64) to chimeric and caninised RA2 and RF2. The binding to CD64 indicates that these antibodies will function via antibody-dependent cellular cytotoxicity (ADCC). All forms of chimeric and caninised antibodies based on the HCB and HCC isotypes were positive in this assay.

### Example 10

### Felinised RA2 and RF2 Antibodies

Given the strong and unexpected binding of RA2 and RF2 to cyclic feline CD20 peptide and the success of design and construction of caninised versions therefrom, felinised and chimeric feline versions of RA2 and RF2 would be desirable for the treatment of feline B-cell mediated diseases. SEQ ID NO:51, 52, 53 and 54 respectively represent felinised variable domains of RA2 and RF2 heavy and light chains respectively. These may be constructed into fully feline versions of RA2 and RF2 antibodies using feline constant domains using the methods illustrated using murine and canine versions above.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. An antibody or an antigen binding fragment thereof that specifically binds to a cyclic polypeptide fragment of CD20 for use in the treatment or prevention of a condition mediated by B-cells in a canine or feline subject in need thereof, wherein the cyclic polypeptide fragment comprises (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide is oxidised by the presence of a disulphide bond formed between the first and second cysteine residues.

2. The antibody or antigen binding fragment thereof as claimed in claim 1 wherein the contiguous amino acid sequence consists of amino acid residues SEKNSL (SEQ ID NO:68).

3. The antibody or antigen binding fragment thereof as claimed in claim 1 wherein the contiguous amino acid sequence consists of amino acid residues PSEKNS (SEQ ID NO:69).

4. The antibody or antigen binding fragment thereof as claimed in claim 1 wherein the contiguous amino acid sequence consists of amino acid residues PSEKNSL (SEQ ID NO 1).

5. The antibody or antigen binding fragment thereof as claimed in claim 4 wherein the cyclic polypeptide fragment consists of SEQ ID NO:2 or an amino acid sequence having at least 85% sequence identity thereto and the subject is a canine subject.

6. The antibody or antigen binding fragment thereof as claimed in claim 4 wherein the cyclic polypeptide fragment consists of SEQ ID NO:4 or an amino acid sequence having at least 85% sequence identity thereto and the subject is a feline subject.

7. The antibody or antigen binding fragment thereof as claimed in any one of claims 1 to 6 wherein the antibody is derived from a Type II anti-human or anti-murine CD20 antibody.

8. The antibody or antigen binding fragment thereof as claimed in claim 7 wherein the Type II anti-human or anti-murine CD20 antibody is selected from the group consisting of B1-H299, GA101 and Bly1.

9. The antibody or antigen binding fragment thereof as claimed in claim 8 wherein the antibody is derived from GA101.

10. The antibody or antigen binding fragment thereof as claimed in claim 9 wherein the subject is a canine and the antibody or antigen binding fragment comprises a light chain variable region comprising at least one of:
an FR1 framework region consisting of or comprising the amino acid sequence of SEQ ID NO:7,
an FR2 framework region consisting of or comprising the amino acid sequence of SEQ ID NO:8,
an FR3 framework region consisting of or comprising the amino acid sequence of SEQ ID NO:9, and
an FR4 framework region consisting of or comprising the amino acid sequence of SEQ ID NO:10,
and/or a heavy chain variable region comprising at least one of:
an FR1 framework region consisting of or comprising the amino acid sequence of SEQ ID NO:11,
an FR2 framework region consisting of or comprising the amino acid sequence of SEQ ID NO:12,
an FR3 framework region consisting of or comprising the amino acid sequence of SEQ ID NO:13, and
an FR4 framework region consisting of or comprising the amino acid sequence of SEQ ID NO:14.

11. The antibody or antigen binding fragment thereof as claimed in claim 10 wherein the antibody or antigen binding fragment thereof comprises a light chain variable region comprising the amino acid sequence of SEQ ID NO:15 or an amino acid sequence which has an identity of at least 85% thereto and/or a heavy chain variable region comprising the amino acid sequence of SEQ ID NO:16 or an amino acid sequence which has an identity of at least 85% thereto.

12. The antibody or antigen binding fragment thereof as claimed in claim 11 wherein the antibody or antigen binding fragment thereof comprises a light chain comprising the amino acid sequence of SEQ ID NO:18 or an amino acid sequence which has an identity of at least 85% thereto and/or a heavy chain comprising the amino acid sequence of SEQ ID NO:17 or an amino acid sequence which has an identity of at least 85% thereto.

13. The antibody or an antigen binding fragment thereof as claimed in any one of claims 1 to 6 wherein the antibody or antigen binding fragment thereof comprises a heavy chain variable region comprising a complementarity determining region 1 (CDR1) region comprising the amino acid sequence of SEQ ID NO:55, a CDR2 region comprising the amino acid sequence of SEQ ID NO:56 and a CDR3 region comprising the amino acid sequence of SEQ ID NO:57, a light chain variable region comprising a CDR1 region comprising the amino acid sequence of SEQ ID NO:58, a CDR2 region comprising the amino acid sequence of SEQ ID NO:59 and a CDR3 region comprising the amino acid sequence of SEQ ID NO:60.

14. The antibody or antigen binding fragment thereof as claimed in claim 13 wherein the subject is a canine and the heavy chain variable region comprises the amino acid sequence of SEQ ID NO:37 or an amino acid sequence which has an identity of at least 85% thereto and/or the light chain variable region comprises the amino acid sequence of SEQ ID NO:38 or an amino acid sequence which has an identity of at least 85% thereto.

15. The antibody or antigen binding fragment thereof as claimed in claim 14 wherein the heavy chain comprises an amino acid sequence selected from the group consisting of SEQ ID NO:39, SEQ ID NO:40, SEQ ID NO:41 and SEQ ID NO:42, or an amino acid sequence which has an identity of at least 85% to SEQ ID NO:39, SEQ ID NO:40, SEQ ID NO:41 or SEQ ID NO:42.

16. The antibody or antigen binding fragment thereof as claimed in claim 14 or 15 wherein the light chain comprises the amino acid sequence of SEQ ID NO:43 or an amino acid sequence which has an identity of at least 85% thereto.

17. The antibody or antigen binding fragment thereof as claimed in claim 13 wherein the subject is a feline and the heavy chain variable region comprises the amino acid sequence of SEQ ID NO:51 or an amino acid sequence which has an identity of at least 85% thereto and/or the light chain variable region comprises the amino acid sequence of SEQ ID NO:52 or an amino acid sequence which has an identity of at least 85% thereto.

18. The antibody or antigen binding fragment thereof as claimed in claim 13 wherein the subject is a canine and wherein the heavy chain comprises an amino acid sequence selected from the group consisting of SEQ ID NO:27, SEQ ID NO:28, SEQ ID NO:29 and SEQ ID NO:30, or an amino acid sequence which has an identity of at least 85% to SEQ ID NO:27, SEQ ID NO:28, SEQ ID NO:29 or SEQ ID NO:30.

19. The antibody or antigen binding fragment thereof as claimed in claim 13 or 18 wherein the subject is a canine and the light chain comprises the amino acid sequence of SEQ ID NO:31 or an amino acid sequence which has an identity of at least 85% thereto.

20. The antibody or an antigen binding fragment thereof as claimed in any one of claims 1 to 6 wherein the antibody or antigen binding fragment thereof comprises a heavy chain variable region comprising a complementarity determining region 1 (CDR1) region comprising the amino acid sequence of SEQ ID NO:61, a CDR2 region comprising the amino acid sequence of SEQ ID NO:62 and a CDR3 region comprising the amino acid sequence of SEQ ID NO:63, a light chain variable region comprising a CDR1 region comprising the amino acid sequence of SEQ ID NO:64, a CDR2 region comprising the amino acid sequence of SEQ ID NO:65 and a CDR3 region comprising the amino acid sequence of SEQ ID NO:66.

21. The antibody or antigen binding fragment thereof as claimed in claim 20 wherein the subject is a canine and the heavy chain variable region comprises the amino acid sequence of SEQ ID NO:44 or an amino acid sequence which has an identity of at least 85% thereto and/or the light chain variable region comprises the amino acid sequence of SEQ ID NO:45 or an amino acid sequence which has an identity of at least 85% thereto.

22. The antibody or antigen binding fragment thereof as claimed in claim 21 wherein the heavy chain comprises an amino acid sequence selected from the group consisting of SEQ ID NO:46, SEQ ID NO:47, SEQ ID NO:48 and SEQ ID NO:49, or an amino acid sequence which has an identity of at least 85% to SEQ ID NO:46, SEQ ID NO:47, SEQ ID NO:48 or SEQ ID NO:49.

23. The antibody or antigen binding fragment thereof as claimed in claim 21 or 22 wherein the light chain comprises the amino acid sequence of SEQ ID NO:50 or an amino acid sequence which has an identity of at least 85% thereto.

24. The antibody or antigen binding fragment thereof as claimed in claim 20 wherein the subject is a feline and the heavy chain variable region comprises the amino acid sequence of SEQ ID NO:53 or an amino acid sequence which has an identity of at least 85% thereto and/or the light chain variable region comprises the amino acid sequence of SEQ ID NO:54 or an amino acid sequence which has an identity of at least 85% thereto.

25. The antibody or antigen binding fragment thereof as claimed in claim 20 wherein the subject is a canine and wherein the heavy chain comprises an amino acid sequence selected from the group consisting of SEQ ID NO:32, SEQ ID NO:33, SEQ ID NO:34 and SEQ ID NO:35, or an amino acid sequence which has an identity of at least 85% to SEQ ID NO:32, SEQ ID NO:33, SEQ ID NO:34 or SEQ ID NO:35.

26. The antibody or antigen binding fragment thereof as claimed in claim 20 or 25 wherein the subject is a canine and wherein the light chain comprises the amino acid sequence of SEQ ID NO:36 or an amino acid sequence which has an identity of at least 85% thereto.

27. The antibody or antigen binding fragment thereof as claimed in any one of claims 1 to 26 wherein the condition mediated by B-cells is a CD20+ B cell lymphoma.

28. The antibody or antigen binding fragment thereof as claimed in any one of claims 1 to 26 wherein the condition mediated by B-cells is an immune mediated disease.

29. The antibody or antigen binding fragment thereof as claimed in claim 28 wherein the immune mediated disease is an autoimmune disease.

30. The antibody or antigen binding fragment thereof as claimed in claim 29 wherein the autoimmune disease is selected from the group consisting of rheumatoid arthritis, systemic lupus erythematosus (SLE), Sjogren's syndrome, vasculitis, multiple sclerosis, Graves' disease, idiopathic thrombocytopenia, dermatomyositis, immune mediated thrombocytopenia, polymyocytosis, pemphigus, immune mediated haemolytic anaemia and bullous pemphigoid.

31. A method for treating or preventing a condition mediated by B-cells in a canine or feline subject in need thereof, the method comprising the step of administering a therapeutically effective amount of an antibody or an antigen binding fragment thereof that specifically binds to a cyclic polypeptide fragment of CD20, wherein the cyclic polypeptide fragment comprises (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide is oxidised by the presence of a disulphide bond formed between the first and second cysteine residues.

32. The method as claimed in claim 31 wherein the contiguous amino acid sequence consists of amino acid residues SEKNSL (SEQ ID NO:68).

33. The method as claimed in claim 31 wherein the contiguous amino acid sequence consists of amino acid residues PSEKNS (SEQ ID NO:69).

34. The method as claimed in claim 31 wherein the contiguous amino acid sequence consists of amino acid residues PSEKNSL (SEQ ID NO 1).

35. The method as claimed in claim 34 wherein the cyclic polypeptide fragment consists of SEQ ID NO:2 or an amino acid sequence having at least 85% sequence identity thereto and the subject is a canine subject.

36. The method as claimed in claim 34 wherein the cyclic polypeptide fragment consists of SEQ ID NO:4 or an amino acid sequence having at least 85% sequence identity thereto and the subject is a feline subject.

37. The method as claimed in any one of claims 31 to 36 wherein the antibody is derived from a Type II anti-human or anti-murine CD20 antibody.

38. The method as claimed in claim 37 wherein the Type II anti-human or anti-murine CD20 antibody is selected from the group consisting of B1-H299, GA101 and Bly1.

39. The method as claimed in claim 38 wherein the antibody is derived from GA101.

40. The method as claimed in claim 39 wherein the subject is a canine and the antibody or antigen binding fragment comprises a light chain variable region comprising at least one of:
an FR1 framework region consisting of or comprising the amino acid sequence of SEQ ID NO:7,
an FR2 framework region consisting of or comprising the amino acid sequence of SEQ ID NO:8,
an FR3 framework region consisting of or comprising the amino acid sequence of SEQ ID NO:9, and
an FR4 framework region consisting of or comprising the amino acid sequence of SEQ ID NO:10,
and/or a heavy chain variable region comprising at least one of:
an FR1 framework region consisting of or comprising the amino acid sequence of SEQ ID NO:11,
an FR2 framework region consisting of or comprising the amino acid sequence of SEQ ID NO:12,
an FR3 framework region consisting of or comprising the amino acid sequence of SEQ ID NO:13, and
an FR4 framework region consisting of or comprising the amino acid sequence of SEQ ID NO:14.

41. The method as claimed in claim 40 wherein the antibody or antigen binding fragment thereof comprises a light chain variable region comprising the amino acid sequence of SEQ ID NO:15 or an amino acid sequence which has an identity of at least 85% thereto and/or a heavy chain variable region comprising the amino acid sequence of SEQ ID NO:16 or an amino acid sequence which has an identity of at least 85% thereto.

42. The method as claimed in claim 41 wherein the antibody or antigen binding fragment thereof comprises a light chain comprising the amino acid sequence of SEQ ID NO:18 or an amino acid sequence which has an identity of at least 85% thereto and/or a heavy chain comprising the amino acid sequence of SEQ ID NO:17 or an amino acid sequence which has an identity of at least 85% thereto.

43. The method as claimed in any one of claims 31 to 36 wherein the antibody or antigen binding fragment thereof comprises a heavy chain variable region comprising a complementarity determining region 1 (CDR1) region comprising the amino acid sequence of SEQ ID NO:55, a CDR2 region comprising the amino acid sequence of SEQ ID NO:56 and a CDR3 region comprising the amino acid sequence of SEQ ID NO:57, a light chain variable region comprising a CDR1 region comprising the amino acid sequence of SEQ ID NO:58, a CDR2 region comprising the amino acid sequence of SEQ ID NO:59 and a CDR3 region comprising the amino acid sequence of SEQ ID NO:60.

44. The method as claimed in claim 43 wherein the subject is a canine and the heavy chain variable region comprises the amino acid sequence of SEQ ID NO:37 or an amino acid sequence which has an identity of at least 85% thereto and/or the light chain variable region comprises the amino acid sequence of SEQ ID NO:38 or an amino acid sequence which has an identity of at least 85% thereto.

45. The method as claimed in claim 44 wherein the heavy chain comprises an amino acid sequence selected from the group consisting of SEQ ID NO:39, SEQ ID NO:40, SEQ ID NO:41 and SEQ ID NO:42, or an amino acid sequence which has an identity of at least 85% to SEQ ID NO:39, SEQ ID NO:40, SEQ ID NO:41 or SEQ ID NO:42.

46. The method as claimed in claim 44 or 45 wherein the light chain comprises the amino acid sequence of SEQ ID NO:43 or an amino acid sequence which has an identity of at least 85% thereto.

47. The method as claimed in claim 43 wherein the subject is a feline and the heavy chain variable region comprises the amino acid sequence of SEQ ID NO:51 or an amino acid sequence which has an identity of at least 85% thereto and/or the light chain variable region comprises the amino acid sequence of SEQ ID NO:52 or an amino acid sequence which has an identity of at least 85% thereto.

48. The method as claimed in claim 43 wherein the subject is a canine and wherein the heavy chain comprises an amino acid sequence selected from the group consisting of SEQ ID NO:27, SEQ ID NO:28, SEQ ID NO:29 and SEQ ID NO:30, or an amino acid sequence which has an identity of at least 85% to SEQ ID NO:27, SEQ ID NO:28, SEQ ID NO:29 or SEQ ID NO:30.

49. The method as claimed in claim 43 or 48 wherein the subject is a canine and the light chain comprises the amino acid sequence of SEQ ID NO:31 or an amino acid sequence which has an identity of at least 85% thereto.

50. The method as claimed in any one of claims 31 to 36 wherein the antibody or antigen binding fragment thereof comprises a heavy chain variable region comprising a complementarity determining region 1 (CDR1) region comprising the amino acid sequence of SEQ ID NO:61, a CDR2 region comprising the amino acid sequence of SEQ ID NO:62 and a CDR3 region comprising the amino acid sequence of SEQ ID NO:63, a light chain variable region comprising a CDR1 region comprising the amino acid sequence of SEQ ID NO:64, a CDR2 region comprising the amino acid sequence of SEQ ID NO:65 and a CDR3 region comprising the amino acid sequence of SEQ ID NO:66.

51. The method as claimed in claim 50 wherein the subject is a canine and the heavy chain variable region comprises the amino acid sequence of SEQ ID NO:44 or an amino acid sequence which has an identity of at least 85% thereto and/or the light chain variable region comprises the amino acid sequence of SEQ ID NO:45 or an amino acid sequence which has an identity of at least 85% thereto.

52. The method as claimed in claim 51 wherein the heavy chain comprises an amino acid sequence selected from the group consisting of SEQ ID NO:46, SEQ ID NO:47, SEQ ID NO:48 and SEQ ID NO:49, or an amino acid sequence which has an identity of at least 85% to SEQ ID NO:46, SEQ ID NO:47, SEQ ID NO:48 or SEQ ID NO:49.

53. The method as claimed in claim 51 or 52 wherein the light chain comprises the amino acid sequence of SEQ ID NO:50 or an amino acid sequence which has an identity of at least 85% thereto.

54. The method as claimed in claim 50 wherein the subject is a feline and the heavy chain variable region comprises the amino acid sequence of SEQ ID NO:53 or an amino acid sequence which has an identity of at least 85% thereto and/or the light chain variable region comprises the amino acid sequence of SEQ ID NO:54 or an amino acid sequence which has an identity of at least 85% thereto.

55. The method as claimed in claim 50 wherein the subject is a canine and wherein the heavy chain comprises an amino acid sequence selected from the group consisting of SEQ ID NO:32, SEQ ID NO:33, SEQ ID NO:34 and SEQ ID NO:35, or an amino acid sequence which has an identity of at least 85% to SEQ ID NO:32, SEQ ID NO:33, SEQ ID NO:34 or SEQ ID NO:35.

56. The method as claimed in claim 50 or 55 wherein the subject is a canine and wherein the light chain comprises the amino acid sequence of SEQ ID NO:36 or an amino acid sequence which has an identity of at least 85% thereto.

57. The method as claimed in any one of claims 31 to 56 wherein the condition mediated by B-cells is a CD20+ B cell lymphoma.

58. The method as claimed in any one of claims 31 to 56 wherein the condition mediated by B-cells is an immune mediated disease.

59. The method as claimed in claim 58 wherein the immune mediated disease is an autoimmune disease.

60. The method as claimed in claim 59 wherein the autoimmune disease is selected from the group consisting of rheumatoid arthritis, systemic lupus erythematosus (SLE), Sjogren's syndrome, vasculitis, multiple sclerosis, Graves' disease, idiopathic thrombocytopenia, dermatomyositis, immune mediated thrombocytopenia, polymyocytosis, pemphigus, immune mediated haemolytic anaemia and bullous pemphigoid.

61. Use of an antibody or an antigen binding fragment thereof that specifically binds to a cyclic polypeptide fragment of CD20 in the preparation of a medicament for the treatment or prevention of a condition mediated by B-cells in a canine or feline subject in need thereof, wherein the cyclic polypeptide fragment comprises (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide is oxidised by the presence of a disulphide bond formed between the first and second cysteine residues.

62. The use as claimed in claim 61 wherein the contiguous amino acid sequence consists of amino acid residues SEKNSL (SEQ ID NO:68).

63. The use as claimed in claim 61 wherein the contiguous amino acid sequence consists of amino acid residues PSEKNS (SEQ ID NO:69).

64. The use as claimed in claim 61 wherein the contiguous amino acid sequence consists of amino acid residues PSEKNSL (SEQ ID NO 1).

65. The use as claimed in claim 64 wherein the cyclic polypeptide fragment consists of SEQ ID NO:2 or an amino acid sequence having at least 85% sequence identity thereto and the subject is a canine subject.

66. The use as claimed in claim 64 wherein the cyclic polypeptide fragment consists of SEQ ID NO:4 or an amino acid sequence having at least 85% sequence identity thereto and the subject is a feline subject.

67. The use as claimed in any one of claims 61 to 66 wherein the antibody is derived from a Type II anti-human or anti-murine CD20 antibody.

68. The use as claimed in claim 67 wherein the Type II anti-human or anti-murine CD20 antibody is selected from the group consisting of B1-H299, GA101 and Bly1.

69. The use as claimed in claim 68 wherein the antibody is derived from GA101.

70. The use as claimed in claim 69 wherein the subject is a canine and the antibody or antigen binding fragment comprises a light chain variable region comprising at least one of:
an FR1 framework region consisting of or comprising the amino acid sequence of SEQ ID NO:7,
an FR2 framework region consisting of or comprising the amino acid sequence of SEQ ID NO:8,
an FR3 framework region consisting of or comprising the amino acid sequence of SEQ ID NO:9, and
an FR4 framework region consisting of or comprising the amino acid sequence of SEQ ID NO:10,
and/or a heavy chain variable region comprising at least one of:
an FR1 framework region consisting of or comprising the amino acid sequence of SEQ ID NO:11,
an FR2 framework region consisting of or comprising the amino acid sequence of SEQ ID NO:12,
an FR3 framework region consisting of or comprising the amino acid sequence of SEQ ID NO:13, and
an FR4 framework region consisting of or comprising the amino acid sequence of SEQ ID NO:14.

71. The use as claimed in claim 70 wherein the antibody or antigen binding fragment thereof comprises a light chain variable region comprising the amino acid sequence of SEQ ID NO:15 or an amino acid sequence which has an identity of at least 85% thereto and/or a heavy chain variable region comprising the amino acid sequence of SEQ ID NO:16 or an amino acid sequence which has an identity of at least 85% thereto.

72. The use as claimed in claim 71 wherein the antibody or antigen binding fragment thereof comprises a light chain comprising the amino acid sequence of SEQ ID NO:18 or an amino acid sequence which has an identity of at least 85% thereto and/or a heavy chain comprising the amino acid sequence of SEQ ID NO:17 or an amino acid sequence which has an identity of at least 85% thereto.

73. The use as claimed in any one of claims 61 to 66 wherein the antibody or antigen binding fragment thereof comprises a heavy chain variable region comprising a complementarity determining region 1 (CDR1) region comprising the amino acid sequence of SEQ ID NO:55, a CDR2 region comprising the amino acid sequence of SEQ ID NO:56 and a CDR3 region comprising the amino acid sequence of SEQ ID NO:57, a light chain variable region comprising a CDR1 region comprising the amino acid sequence of SEQ ID NO:58, a CDR2 region comprising the amino acid sequence of SEQ ID NO:59 and a CDR3 region comprising the amino acid sequence of SEQ ID NO:60.

74. The use as claimed in claim 73 wherein the subject is a canine, the heavy chain variable region comprises the amino acid sequence of SEQ ID NO:37 or an amino acid sequence which has an identity of at least 85% thereto and/or the light chain variable region comprises the amino acid sequence of SEQ ID NO:38 or an amino acid sequence which has an identity of at least 85% thereto.

75. The use as claimed in claim 74 wherein the heavy chain comprises an amino acid sequence selected from the group consisting of SEQ ID NO:39, SEQ ID NO:40, SEQ ID NO:41 and SEQ ID NO:42, or an amino acid sequence which has an identity of at least 85% to SEQ ID NO:39, SEQ ID NO:40, SEQ ID NO:41 or SEQ ID NO:42.

76. The use as claimed in claim 74 or 75 wherein the light chain comprises the amino acid sequence of SEQ ID NO:43 or an amino acid sequence which has an identity of at least 85% thereto.

77. The use as claimed in claim 73 wherein the subject is a feline and the heavy chain variable region comprises the amino acid sequence of SEQ ID NO:51 or an amino acid sequence which has an identity of at least 85% thereto and/or the light chain variable region comprises the amino acid sequence of SEQ ID NO:52 or an amino acid sequence which has an identity of at least 85% thereto.

78. The use as claimed in claim 73 wherein the subject is a canine and wherein the heavy chain comprises an amino acid sequence selected from the group consisting of SEQ ID NO:27, SEQ ID NO:28, SEQ ID NO:29 and SEQ ID NO:30, or an amino acid sequence which has an identity of at least 85% to SEQ ID NO:27, SEQ ID NO:28, SEQ ID NO:29 or SEQ ID NO:30.

79. The use as claimed in claim 73 or 78 wherein the subject is a canine and the light chain comprises the amino acid sequence of SEQ ID NO:31 or an amino acid sequence which has an identity of at least 85% thereto.

80. The use as claimed in any one of claims 61 to 66 wherein the antibody or antigen binding fragment thereof comprises a heavy chain variable region comprising a complementarity determining region 1 (CDR1) region comprising the amino acid sequence of SEQ ID NO:61, a CDR2 region comprising the amino acid sequence of SEQ ID NO:62 and a CDR3 region comprising the amino acid sequence of SEQ ID NO:63, a light chain variable region comprising a CDR1 region comprising the amino acid sequence of SEQ ID NO:64, a CDR2 region comprising the amino acid sequence of SEQ ID NO:65 and a CDR3 region comprising the amino acid sequence of SEQ ID NO:66.

81. The use as claimed in claim 80 wherein the subject is a canine and the heavy chain variable region comprises the amino acid sequence of SEQ ID NO:44 or an amino acid sequence which has an identity of at least 85% thereto and/or the light chain variable region comprises the amino acid sequence of SEQ ID NO:45 or an amino acid sequence which has an identity of at least 85% thereto.

82. The use as claimed in claim 81 wherein the heavy chain comprises an amino acid sequence selected from the group consisting of SEQ ID NO:46, SEQ ID NO:47, SEQ ID NO:48 and SEQ ID NO:49, or an amino acid sequence which has an identity of at least 85% to SEQ ID NO:46, SEQ ID NO:47, SEQ ID NO:48 or SEQ ID NO:49.

83. The use as claimed in claim 81 or 82 wherein the light chain comprises the amino acid sequence of SEQ ID NO:50 or an amino acid sequence which has an identity of at least 85% thereto.

84. The use as claimed in claim 80 wherein the subject is a feline and the heavy chain variable region comprises the amino acid sequence of SEQ ID NO:53 or an amino acid sequence which has an identity of at least 85% thereto and/or the light chain variable region comprises the amino acid sequence of SEQ ID NO:54 or an amino acid sequence which has an identity of at least 85% thereto.

85. The use as claimed in claim 80 wherein the subject is a canine and wherein the heavy chain comprises an amino acid sequence selected from the group consisting of SEQ ID NO:32, SEQ ID NO:33, SEQ ID NO:34 and SEQ ID NO:35, or an amino acid sequence which has an identity of at least 85% to SEQ ID NO:32, SEQ ID NO:33, SEQ ID NO:34 or SEQ ID NO:35.

86. The use as claimed in claim 80 or 85 wherein the subject is a canine, wherein the light chain comprises the amino acid sequence of SEQ ID NO:36 or an amino acid sequence which has an identity of at least 85% thereto.

87. The use as claimed in any one of claims 61 to 86 wherein the condition mediated by B-cells is a CD20+ B cell lymphoma.

88. The use as claimed in any one of claims 61 to 86 wherein the condition mediated by B-cells is an immune mediated disease.

89. The use as claimed in claim 88 wherein the immune mediated disease is an autoimmune disease.

90. The use as claimed in claim 89 wherein the autoimmune disease is selected from the group consisting of rheumatoid arthritis, systemic lupus erythematosus (SLE), Sjogren's syndrome, vasculitis, multiple sclerosis, Graves' disease, idiopathic thrombocytopenia, dermatomyositis, immune mediated thrombocytopenia, polymyocytosis, pemphigus, immune mediated haemolytic anaemia and bullous pemphigoid.

91. A caninised or felinised antibody or an antigen binding fragment thereof which binds specifically to a cyclic polypeptide fragment of CD20, wherein the cyclic polypeptide fragment comprises (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide is oxidised by the presence of a disulphide bond formed between the first and second cysteine residues.

92. The antibody or antigen binding fragment thereof as claimed in claim 91 wherein the antibody is a caninised antibody comprising complementarity determining regions of a heavy and/or light chain from a donor antibody from a species other than a canine, wherein the donor antibody has binding specificity for the cyclic polypeptide fragment.

93. The antibody or antigen binding fragment thereof as claimed in claim 92 wherein the antibody comprises framework regions of the heavy and/or light chain from the donor antibody.

94. The antibody or antigen binding fragment thereof as claimed in claim 93 wherein the framework regions of the heavy and/or light chain from the donor antibody are modified to substitute amino acid residues that are foreign at a corresponding position in canine antibodies with amino acid residues present at the corresponding position in canine antibodies.

95. The antibody or antigen binding fragment thereof as claimed in claim 91 wherein the antibody is a felinised antibody comprising complementarity determining regions of a heavy and/or light chain from a donor antibody from a species other than a feline, wherein the donor antibody has binding specificity for the cyclic polypeptide fragment.

96. The antibody or antigen binding fragment thereof as claimed in claim 95 wherein the antibody comprises framework regions of the heavy and/or light chain from the donor antibody.

97. The antibody or antigen binding fragment thereof as claimed in claim 96 wherein the framework regions of the heavy and/or light chain from the donor antibody are modified to substitute amino acid residues that are foreign at a corresponding position in feline antibodies with amino acid residues present at the corresponding position in feline antibodies.

98. The antibody or antigen binding fragment thereof as claimed in claim 94 or 97 wherein the amino acid residues that are foreign at the corresponding position in canine or feline antibodies are substituted with the amino acid residues present at the corresponding position which have the highest homology to the substituted amino acid residues.

99. The antibody or antigen binding fragment thereof as claimed in any one of claims 91 to 99 wherein the antibody or antigen binding fragment comprises constant domains of a heavy and/or light chain from a canine or feline antibody.

100. The antibody or antigen binding fragment thereof as claimed in any one of claims 93 to 99 wherein the donor antibody is a Type II anti-human or anti-murine CD20 antibody.

101. The antibody or antigen binding fragment thereof as claimed in claim 100 wherein the Type II anti-human or anti-murine CD20 antibody is selected from the group consisting of B1-H299, GA101 and Bly1.

102. A humanised antibody or an antigen binding fragment thereof which binds specifically to a cyclic polypeptide fragment of CD20, wherein the cyclic polypeptide fragment comprises (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide is oxidised by the presence of a disulphide bond formed between the first and second cysteine residues, and wherein framework regions of the heavy and/or light chain are derived from an antibody obtained from a species other than human and the framework regions are modified to substitute amino acid residues that are foreign at a corresponding position in human antibodies with amino acid residues present at the corresponding position in human antibodies.

103. The antibody or antigen binding fragment thereof as claimed in claim 102 wherein the amino acid residues that are foreign at the corresponding position in human antibodies are substituted with the amino acid residues present at the corresponding position which have the highest homology to the one or more substituted amino acid residues.

104. A chimeric antibody or an antigen binding fragment thereof which binds specifically to a cyclic polypeptide fragment of CD20, wherein the cyclic polypeptide fragment comprises (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide is oxidised by the presence of a disulphide bond formed between the first and second cysteine residues and wherein the antibody comprises a canine or feline constant domain.

105. An antibody or an antigen binding fragment thereof, which specifically binds to CD20 wherein the antibody or fragment thereof comprises a heavy chain variable region comprising a complementarity determining region 1 (CDR1) region comprising the amino acid sequence of SEQ ID NO:55, a CDR2 region comprising the amino acid sequence of SEQ ID NO:56 and a CDR3 region comprising the amino acid sequence of SEQ ID NO:57, a light chain variable region comprising a CDR1 region comprising the amino acid sequence of SEQ ID NO:58, a CDR2 region comprising the amino acid sequence of SEQ ID NO:59 and a CDR3 region comprising the amino acid sequence of SEQ ID NO:60.

106. The antibody or antigen binding fragment thereof as claimed in claim 105 wherein the heavy chain variable region comprises the amino acid sequence of SEQ ID NO:19 or an amino acid sequence which has an identity of at least 85% thereto and/or the light chain variable region comprises the amino acid sequence of SEQ ID NO:20 or an amino acid sequence which has an identity of at least 85% thereto.

107. The antibody or antigen binding fragment thereof as claimed in claim 106 wherein the heavy chain comprises the amino acid sequence of SEQ ID NO:23 or an amino acid sequence which has an identity of at least 85% thereto and/or the light chain comprises the amino acid sequence of SEQ ID NO:24 or an amino acid sequence which has an identity of at least 85% thereto.

108. The antibody or antigen binding fragment thereof as claimed in claim 105 wherein the antibody is a caninised antibody.

109. The antibody or antigen binding fragment thereof as claimed in claim 108 wherein the heavy chain variable region comprises the amino acid sequence of SEQ ID NO:37 or an amino acid sequence which has an identity of at least 85% thereto and/or the light chain variable region comprises the amino acid sequence of SEQ ID NO:38 or an amino acid sequence which has an identity of at least 85% thereto.

110. The antibody or antigen binding fragment thereof as claimed in claim 109 wherein the heavy chain comprises an amino acid sequence selected from the group consisting of SEQ ID NO:39, SEQ ID NO:40, SEQ ID NO:41 and SEQ ID NO:42, or an amino acid sequence which has an identity of at least 85% to SEQ ID NO:39, SEQ ID NO:40, SEQ ID NO:41 or SEQ ID NO:42.

111. The antibody or antigen binding fragment thereof as claimed in claim 109 or 110 wherein the light chain comprises the amino acid sequence of SEQ ID NO:43 or an amino acid sequence which has an identity of at least 85% thereto.

112. The antibody or antigen binding fragment thereof as claimed in claim 105 wherein the antibody is a felinised antibody.

113. The antibody or antigen binding fragment thereof as claimed in claim 112 wherein the heavy chain variable region comprises the amino acid sequence of SEQ ID NO:51 or an amino acid sequence which has an identity of at least 85% thereto and/or the light chain variable region comprises the amino acid sequence of SEQ ID NO:52 or an amino acid sequence which has an identity of at least 85% thereto.

114. The antibody or antigen binding fragment thereof as claimed in claim 105 wherein the antibody is a chimeric antibody.

115. The antibody or antigen binding fragment thereof as claimed in claim 114 wherein the heavy chain and/or light chain comprises a constant domain derived from a canine antibody.

116. The antibody or antigen binding fragment thereof as claimed in claim 115 wherein the heavy chain comprises an amino acid sequence selected from the group consisting of SEQ ID NO:27, SEQ ID NO:28, SEQ ID NO:29 and SEQ ID NO:30, or an amino acid sequence which has an identity of at least 85% to SEQ ID NO:27, SEQ ID NO:28, SEQ ID NO:29 or SEQ ID NO:30.

117. The antibody or antigen binding fragment thereof as claimed in claim 115 or 116 wherein the light chain comprises the amino acid sequence of SEQ ID NO:31 or an amino acid sequence which has an identity of at least 85% thereto.

118. An antibody or an antigen binding fragment thereof, which specifically binds to CD20 wherein the antibody or fragment thereof comprises a heavy chain variable region comprising a complementarity determining region 1 (CDR1) region comprising the amino acid sequence of SEQ ID NO:61, a CDR2 region comprising the amino acid sequence of SEQ ID NO:62 and a CDR3 region comprising the amino acid sequence of SEQ ID NO:63, a light chain variable region comprising a CDR1 region comprising the amino acid sequence of SEQ ID NO:64, a CDR2 region comprising the amino acid sequence of SEQ ID NO:65 and a CDR3 region comprising the amino acid sequence of SEQ ID NO:66.

119. The antibody or antigen binding fragment thereof as claimed in claim 118 wherein the heavy chain variable region comprises the amino acid sequence of SEQ ID NO:21 or an amino acid sequence which has an identity of at least 85% thereto and/or the light chain variable region comprises the amino acid sequence of SEQ ID NO:22 or an amino acid sequence which has an identity of at least 85% thereto.

120. The antibody or antigen binding fragment thereof as claimed in claim 119 wherein the heavy chain comprises the amino acid sequence of SEQ ID NO:25 or an amino acid sequence which has an identity of at least 85% thereto and/or the light chain comprises the amino acid sequence of SEQ ID NO:26 or an amino acid sequence which has an identity of at least 85% thereto.

121. The antibody or antigen binding fragment thereof as claimed in claim 118 wherein the antibody is a caninised antibody.

122. The antibody or antigen binding fragment thereof as claimed in claim 121 wherein the heavy chain variable region comprises the amino acid sequence of SEQ ID NO:44 or an amino acid sequence which has an identity of at least 85% thereto and/or the light chain variable region comprises the amino acid sequence of SEQ ID NO:45 or an amino acid sequence which has an identity of at least 85% thereto.

123. The antibody or antigen binding fragment thereof as claimed in claim 122 wherein the heavy chain comprises an amino acid sequence selected from the group consisting of SEQ ID NO:46, SEQ ID NO:47, SEQ ID NO:48 and SEQ ID NO:49, or an amino acid sequence which has an identity of at least 85% to SEQ ID NO:46, SEQ ID NO:47, SEQ ID NO:48 or SEQ ID NO:49.

124. The antibody or antigen binding fragment thereof as claimed in claim 122 or 123 wherein the light chain comprises the amino acid sequence of SEQ ID NO:50 or an amino acid sequence which has an identity of at least 85% thereto.

125. The antibody or antigen binding fragment thereof as claimed in claim 118 wherein the antibody is a felinised antibody.

126. The antibody or antigen binding fragment thereof as claimed in claim 125 wherein the heavy chain variable region comprises the amino acid sequence of SEQ ID NO:53 or an amino acid sequence which has an identity of at least 85% thereto and/or the light chain variable region comprises the amino acid sequence of SEQ ID NO:54 or an amino acid sequence which has an identity of at least 85% thereto.

127. The antibody or antigen binding fragment thereof as claimed in claim 118 wherein the antibody is a chimeric antibody.

128. The antibody or antigen binding fragment thereof as claimed in claim 127 wherein the heavy chain and/or light chain comprises a constant domain derived from a canine antibody.

129. The antibody or antigen binding fragment thereof as claimed in claim 128 wherein the heavy chain comprises an amino acid sequence selected from the group consisting of SEQ ID NO:32, SEQ ID NO:33, SEQ ID NO:34 and SEQ ID NO:35, or an amino acid sequence which has an identity of at least 85% to SEQ ID NO:32, SEQ ID NO:33, SEQ ID NO:34 or SEQ ID NO:35.

130. The antibody or antigen binding fragment thereof as claimed in claim 128 or 129 wherein the light chain comprises the amino acid sequence of SEQ ID NO:36 or an amino acid sequence which has an identity of at least 85% thereto.

131. The antibody or antigen binding fragment thereof as claimed in any one of claims 105 to 130 wherein the antibody or antigen binding fragment is cross-reactive and binds to human, canine, murine and feline CD20.

132. An antibody or an antigen binding fragment thereof wherein the antibody or antigen binding fragment thereof comprises a heavy chain variable region comprising the amino acid sequence of SEQ ID NO:16 or an amino acid sequence which has an identity of at least 85% thereto and/or a light chain variable region comprising the amino acid sequence of SEQ ID NO:15 or an amino acid sequence which has an identity of at least 85% thereto.

133. The antibody or antigen binding fragment thereof as claimed in claim 132 wherein the heavy chain comprises the amino acid sequence of SEQ ID NO:17 or an amino acid sequence which has an identity of at least 85% thereto and/or the light chain comprises the amino acid sequence of SEQ ID NO:18 or an amino acid sequence which has an identity of at least 85% thereto.

134. An isolated nucleic acid that encodes an antibody or antigen binding fragment according to any one of claims 105 to 133.

135. An expression vector comprising a nucleic acid as claimed in claim 134.

136. A host cell incorporating the expression vector as claimed in claim 135.

137. A method for producing an antibody comprising the step of culturing a host cell as claimed in claim 136 to allow the cell to express the antibody.

138. An antibody or antigen binding fragment as claimed in any one of claims 91 to 133 for use in the treatment or prevention of a condition mediated by B-cells.

139. A method for treating or preventing a condition mediated by B-cells comprising the steps of administering a therapeutically effective amount of an antibody or antigen binding fragment as claimed in any one of claims 91 to 133 to a subject in need thereof.

140. Use of an antibody or antigen binding fragment as claimed in any one of claims 91 to 133 in the preparation of a medicament for the treatment or prevention of a condition mediated by B-cells.

141. An antibody or antigen binding fragment as claimed in any one of claims 91 to 133 for use in diagnosis.

142. A cyclic polypeptide fragment of CD20 comprising (i) a contiguous amino acid sequence consisting of amino acid residues SEKNS (SEQ ID NO:67); (ii) a first cysteine residue which is present at a region N-terminal to the contiguous amino acid sequence and (iii) a second cysteine residue which is present at a region C-terminal to the contiguous amino acid sequence, wherein the cyclic polypeptide is oxidised by the presence of a disulphide bond formed between the first and second cysteine residues.

143. The cyclic polypeptide fragment as claimed in claim 142 wherein the contiguous amino acid sequence consists of amino acid residues SEKNSL (SEQ ID NO:68).

144. The cyclic polypeptide fragment as claimed in claim 142 wherein the contiguous amino acid sequence consists of amino acid residues PSEKNS (SEQ ID NO:69).

145. The cyclic polypeptide fragment as claimed in claim 142 wherein the contiguous amino acid sequence consists of amino acid residues PSEKNSL (SEQ ID NO 1).

146. The cyclic polypeptide fragment as claimed in any one of claims 142 to 145 wherein the cyclic polypeptide fragment comprises less than 25 amino acid residues.

147. The cyclic polypeptide fragment as claimed in claim 146 wherein the cyclic polypeptide fragment comprises less than 22 amino acid residues.

148. The cyclic polypeptide fragment as claimed in claim 145 wherein the cyclic polypeptide fragment consists of SEQ ID NO:2 or an amino acid sequence having at least 85% sequence identity thereto.

149. The cyclic polypeptide fragment as claimed in claim 145 wherein the cyclic polypeptide fragment consists of SEQ ID NO:4 or an amino acid sequence having at least 85% sequence identity thereto.

150. The cyclic polypeptide fragment as claimed in claim 144 wherein the cyclic polypeptide fragment consists of SEQ ID NO:3 or an amino acid sequence having at least 85% sequence identity thereto.

151. The cyclic polypeptide fragment as claimed in claim 142 wherein the cyclic polypeptide fragment consists of SEQ ID NO:6 or an amino acid sequence having at least 85% sequence identity thereto.

152. The cyclic polypeptide fragment as claimed in any one of claims 142 to 151 wherein binding of the cyclic polypeptide fragment by an antagonistic binding member antagonises CD20 biological activity.

153. A pharmaceutical composition comprising the cyclic polypeptide fragment as claimed in any one of claims 142 to 152 and a pharmaceutically acceptable carrier or excipient.

154. A vaccine composition comprising the cyclic polypeptide fragment as claimed in any one of claims 142 to 152.

155. Use of the cyclic polypeptide fragment as claimed in any one of claims 142 to 152 in a method for generating a binding member which specifically binds to CD20.

156. The use as claimed in claim 155 wherein the method is a method for generating a binding member which specifically binds to canine CD20.

157. The use as claimed in claim 155 wherein the method is a method for generating a binding member which specifically binds to feline CD20.

158. A method for generating a binding member which specifically binds to CD20, the method comprising the steps of:
administering to a subject a cyclic polypeptide fragment as claimed in any one of claims 142 to 152, and
isolating binding agents which bind specifically to said polypeptide.

159. The method as claimed in claim 158 wherein the method is a method for generating a binding member which specifically binds to canine CD20.

160. The method as claimed in claim 158 wherein the method is a method for generating a binding member which specifically binds to feline CD20.

161. A screening method for identifying a binding member which specifically binds to canine and/or feline CD20, the screening method comprising the steps of:
bringing a candidate compound into contact with the cyclic polypeptide fragment as claimed in any one of claims 142 to 152; and
assessing binding between the candidate compound and the cyclic polypeptide fragment as claimed in any one of claims 142 to 152;
wherein binding between the candidate compound and the cyclic polypeptide fragment as claimed in any one of claims 142 to 152 identifies the candidate compound as a binding member which specifically binds to canine and/or feline CD20.

162. A method for detecting the presence of canine or feline CD20 in a B-lymphocyte-containing sample comprising the steps of:
contacting one or more antibodies as claimed in any one of claims 91 to 133 with the sample under conditions that allow B-lymphocyte/antibody complexes to form; and
detecting B-lymphocyte/antibody complexes, wherein the detection of said complexes is an indication that canine or feline CD20 is present in the sample.
